(19) United States
(12) Patent Application Publication
(10) Pub. No.: US 2025/0282890 A1
(43) Pub. Date: Sep. 11, 2025

(54) HUMANIZED ANTI-PSMA ANTIBODY

(71) Applicants: Technische Universität München, München (DE); Institute of Biotechnology CAS, V.V.I., Vestec (CZ)

(72) Inventors: Zora Nováková, Praha (CZ); Cyril Barinka, Praha (CZ); Arne Skerra, Dachau (DE); Sárka Pechoucková, Praha (CZ)

(21) Appl. No.: 18/288,871

(22) PCT Filed: May 12, 2022
(86) PCT No.: PCT/EP2022/062868

§ 371 Date: Oct. 30, 2023

(30) Foreign Application Priority Data
May 12, 2021 (EP) 21173577.4

(51) Int. Cl. C07K 16/30

(52) U.S. Cl. CPC C07K 16/3069; C07K 2317/24; C07K 2317/55; C07K 2317/732; C07K 2317/92

(57) ABSTRACT

The present invention relates to antibodies that bind to prostate-specific membrane antigen (PSMA).

### RELATED APPLICATIONS

This application is a § 371 national-stage application based on International Patent Application No.: PCT/EP2022/062868, filed May 12, 2022, which claims the benefit of priority from European Application No.: 21173577.4, filed on May 12, 2021. The contents of PCT/EP2022/062868 are incorporated by reference herein in their entirety.

The present invention relates to an antibody binding to prostate-specific membrane antigen (PSMA), wherein the antibody comprises the V_{H} region determined by the amino acid sequence of EVQLVESGGGLVQPGGSLRLSCAASGYAF(X₁)(X₂)(X₃)W(X₄)NWVRQAPGKGLEWISRIYPG(X₅)(X₆)(X₇)(X₈)NY(X₉)(X₁₀)KFKGKATISADKSKNTLYLQMNSLRAEDTAVYYCARGEWYLYYFDYWGQGTLVT VSS (SEQ ID NO: 30), wherein for (X₁) to (X₃) at least one applies: (X₁) is G, A, V, L, I, Y, H, K, R, Q, N, E, D, S or T, preferably G, A, Y, H, K, R, Q, N, E, D, S or T, more N or Q, and most preferably Q, (X₂) is P or T, and (X₃) is G, A, V, L, I, Y, H, K, R, Q, N, E, D, T or S and is preferably S, with the proviso that (X₁) to (X₃) are not N-(Z)-S/T, wherein (Z) can be any amino acid but not P, or wherein (X₁) to (X₃) are N-T-S; (X₄) is M, I or L and preferably M; for (X₅) and (X₆) at least one applies: (X₅) is G, A, V, L, I, Y, H, Q, N, E, D, S or T and preferably D or E, and (X₆) is G, A, V, L, I, H, K, R, Q, N, E, D, S or T, preferably G or A, with the proviso that (X₅)-(X₆) are not D-S, N-G or N-S; for (X₇) and (X₈) at least one applies: (X₇) is G, A, V, L, I, Y, H, Q, N, E, D, S or T, preferably D, E or S, and (X₈) is G, A, V, L, H, Q, N, E, D, S or T and preferably T or A, with the proviso that (X₇)-(X₈) are not D-G, D-S, N-G or N-S; for (X₉) and (X₁₀) at least one applies: (X₉) is G, A, V, L, I, Y, H, K, R, Q, N, E, D, S or T and preferably N, E or Q, and (X₁₀) is G, A, V, L, I, Y, H, K, R, Q, N, E, D, S or T, preferably G or A, with the proviso that (X₉)-(X₁₀) are not D-G, D-S or N-S; or an amino acid sequence being at least 90%, preferably at least 95% identical thereto; and the V_{L} region determined by the amino acid sequence of DIOMTQSPSSLSASVGDRVTITCSASOGI(X₁₁)(X₁₂)FLTWYOOKPGKALKLLIYYTSSLHSGVPSRFSG SGSGTDYTLTISSLOPEDFATYYCOOYSNLPFTFGOGTKVEIK (SEQ ID NO: 31), wherein for (X₁₁) and (X₁₂) at least one applies: (X₁₁) is G, A, V, L, I, Y, H, K, R, Q, N, E, D, S or T, preferably N, D, E or Q, and (X₁₂) is G, A, V, L, I, Y, H, K, R, Q, N, E, D, S or T and preferably N, with the proviso that (X₁₁)-(X₁₂) are not D-G, D-S, N-G or N-S; or an amino acid sequence being at least 90%, preferably at least 95% identical thereto, provided that the three CDRs of the V_{H} region are determined by the amino acid sequences of GYAF(X₁)(X₂)(X₃)W(X₄)N(SEQ ID NO: 32), wherein for (X₁) to (X₃) at least one applies: (X₁) is G, A, V, L, I, Y, H, K, R, Q, N, E, D, S or T, preferably G, A, Y, H, K, R, Q, N, E, D, S or T, more preferably N or Q, and most preferably Q, (X₂) is P or T, and (X₃) is G, A, V, L, I, Y, H, K, R, Q, N, E, D, T or S and is preferably S, with the proviso that (X₁) to (X₃) are not N-(Z)-S/T, wherein (Z) can be any amino acid but not P, or wherein (X₁) to (X₃) are N-T-S; and (X₄) is M, I or L and preferably M, RIYPG(X₅)(X₆)(X₇)(X₈)NY(X₉)(X₁₀)KFK (SEQ ID NO: 33), wherein for (X₅) and (X₆) at least one applies: (X₅) is G, A, V, L, I, Y, H, Q, N, E, D, S or T and preferably D or E, and (X₆) is G, A, V, L, I, H, K, R, Q, N, E, D, S or T, preferably G or A, with the proviso that (X₅)-(X₆) are not D-S, N-G or N-S; for (X₇) and (X₈) at least one applies: (X₇) is G, A, V, L, I, Y, H, Q, N, E, D, S or T, preferably D, E or S, and (X₈) is G, A, V, L, H, Q, N, E, D, S or T and preferably T or A, with the proviso that (X₇)-(X₈) are not D-G, D-S, N-G or N-S; for (X₉) and (X₁₀) at least one applies: (X₉) is G, A, V, L, I, Y, H, K, R, Q, N, E, D, S or T and preferably N, E or Q, and (X₁₀) is G, A, V, L, I, Y, H, K, R, Q, N, E, D, S or T, preferably G or A, with the proviso that (X₉)-(X₁₀) are not D-G, D-S or N-S; and SEQ ID NO: 5, and the three CDRs of the V_{L} region are determined by the amino acid sequences of SASQGI(X₁₁)(X₁₂)FLT (SEQ ID NO: 34), wherein for (X₁₁) and (X₁₂) at least one applies: (X₁₁) is G, A, V, L, I, Y, H, K, R, Q, N, E, D, S or T, preferably N, D, E or Q, and (X₁₂) is G, A, V, L, I, Y, H, K, R, Q, N, E, D, S or T and preferably N, with the proviso that (X₁₁)-(X₁₂) are not D-G, D-S, N-G or N-S; SEQ ID NO: 7, and SEQ ID NO: 8.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The increasing length of the human lifespan brings about higher incidence of various health problems including cancer. Prostate cancer (PCa) holds a prominent position among tumor-associated diseases among elderly men in industrial countries due to its high incidence and associated mortality rates of metastatic disease. To improve PCa prognosis, a combination of precise diagnosis of the tumor stage and specifically designed treatment plans is required. In the clinic today, PCa is routinely detected using imaging techniques such as bone scintigraphy, computed tomography, ultrasound, positron emission tomography, single photon emission computed tomography, and magnetic resonance imaging. However, there is still an untapped potential in PCa diagnostics and staging in terms of sensitivity and specificity. Thus, new PCa-specific reagents are extensively researched.

Prostate-specific membrane antigen (PSMA) is one of the leading PCa-specific biomarkers strongly expressed on PCa cells. The specific presence of PSMA in primary, high-grade, androgen-independent, and metastatic PCa tumors predetermines the enzyme as a prime tool for PCa imaging and therapy. Consequently, PSMA-specific antibodies are being developed as new PCa-targeting theranostics. Various constructs have already been tested in vitro on PSMA-expressing cells as well as in vivo in relevant xenograft models. Notably, PCa imaging in human patients has also been performed using a PSMA-specific 89Zr-IAB2M minibody.

Several PSMA-targeting entities are being developed as PCa therapeutics. Site-specific delivery of Notch1-specific siRNA by an anti-PSMA scFv was documented to inhibit PCa tumor growth. Multimeric structures derived from anti-PSMA scFv and bispecific molecules, namely anti-PSMA/anti-CD3 fusions, have been used in preclinical immunotherapy models. Immunotoxin fusions derived from PSMA-specific scFvs showed efficient control of PCa xenografts and enhanced anti-tumor activity of cytotoxic drugs. Interestingly, PSMA-specific antibody fragments were documented to direct the cytotoxicity of chimeric antigen receptor T cells (CAR-T cells) not only to PCa but also to ovarian cancer. In virotherapy approaches, oncolytic viruses targeted to cancer cells via anti-PSMA scFv revealed potential to induce PCa regression (Novakova et al. (2020), Int J Mol Sci. 2020 September; 21(18): 6672).

One very promising candidate of a PSMA-specific antibody for the development of PCa diagnostics and therapeutics is the anti-PSMA antibody 5D3 (Novakova et al. (2017), Prostate. 2017; 77:749-764 and WO 2018/129284). The superior features of 5D3 mAb as compared to other anti-PSMA antibodies include sub-nanomolar affinity and a high specificity for native PSMA. A yet further advantage of the 5D3 mAb is that antibody fragments in the format of single-chain Fv (scFv) and Fab fragments retain the nanomolar affinity and single target specificity of the parent 5D3 antibody (Novakova et al. (2020), Int J Mol Sci. 2020 September; 21(18): 6672). These characteristics make the 5D3 mAb and its fragments particularly suitable for in vivo applications.

However, 5D3 mAb is a murine antibody. It is well-known that the use of murine antibodies in order to fight several diseases (e. g. cancer, rejection after transplantation, rheumatic diseases and autoimmune diseases) did not deliver the expected therapeutic success. The immune response initiated by murine antibodies is one of the main problems. The human immune system recognizes murine antibodies from mice or rats as foreign substances and starts the production of antibodies against these by itself. This leads on one hand to ineffectiveness of the applied murine antibodies and on the other hand to unpleasant side effects like an anaphylactic shock or the serum sickness.

The humanization of murine antibodies which results in antibodies being similar to those of humans is a well-established procedure in order to avoid the unfavourable immune response. Humanization of murine monoclonal antibodies has vastly improved their in vivo tolerability. Humanization is the replacement of the mouse constant regions and V framework regions for human sequences and results in a significantly less immunogenic product (Harding et al. (2010), MAbs. 2010 May-June; 2(3):256-265).

However, it is known and also turned out in connection with the attempt of humanizing 5D3 mAb that it is challenging to obtain a humanized version of a monoclonal murine antibody which fully retains the favorable properties of the monoclonal murine parent antibody. This issue is also well documented in the present invention, where despite our detailed knowledge of the three-dimensional structure of murine 5D3, only a single humanized 5D3 version, exemplified by 5D3-6, met all criteria for successful humanized clone suitable for further development. This challenging task is solved by the antibody of the present invention.

The present invention therefore relates in a first aspect to an antibody binding to prostate-specific membrane antigen (PSMA), wherein the antibody comprises the V_{H} region determined by the amino acid sequence of EVQLVESGGGLVQPGGSLRLSCAASGYAF(X₁)(X₂)(X₃)W(X₄)NWVRQAPGKGLEWISRIYPG(X₅)(X₆)(X₇)(X₈)NY(X₉)(X₁₀)KFKGKATISADKSKNTLYLQMNSLRAEDTAVYYCARGEWYLYYFDYWGQGTLVT VSS (SEQ ID NO: 30), wherein for (X₁) to (X₃) at least one applies: (X₁) is G, A, V, L, I, Y, H, K, R, Q, N, E, D, S or T, preferably G, A, Y, H, K, R, Q, N, E, D, S or T, more preferably N or Q, and most preferably Q, (X₂) is P or T, and (X₃) is G, A, V, L, I, Y, H, K, R, Q, N, E, D, T or S and is preferably S, with the proviso that (X₁) to (X₃) are not N-(Z)-S/T, wherein (Z) can be any amino acid but not P, or wherein (X₁) to (X₃) are N-T-S; (X₄) is M, I or L and preferably M; for (X₅) and (X₆) at least one applies: (X₅) is G, A, V, L, I, Y, H, Q, N, E, D, S or T and preferably D or E, and (X₆) is G, A, V, L, I, H, K, R, Q, N, E, D, S or T, preferably G or A, with the proviso that (X₅)-(X₆) are not D-S, N-G or N-S; for (X₇) and (X₈) at least one applies: (X₇) is G, A, V, L, I, Y, H, Q, N, E, D, S or T, preferably D, E or S, and (X₈) is G, A, V, L, H, Q, N, E, D, S or T and preferably T or A, with the proviso that (X₇)-(X₈) are not D-G, D-S, N-G or N-S; for (X₉) and (X₁₀) at least one applies: (X₉) is G, A, V, L, I, Y, H, K, R, Q, N, E, D, S or T and preferably N, E or Q, and (X₁₀) is G, A, V, L, I, Y, H, K, R, Q, N, E, D, S or T, preferably G or A, with the proviso that (X₉)-(X₁₀) are not D-G, D-S or N-S; or an amino acid sequence being at least 90%, preferably at least 95% identical thereto; and the V_{L} region determined by the amino acid sequence of DIQMTQSPSSLSASVGDRVTITCSASOGI(X₁₁)(X₁₂)FLTWYQQKPGKALKLLIYYTSSLHSGVPSRFSG SGSGTDYTLTISSLQPEDFATYYCQQYSNLPFTFGQGTKVEIK (SEQ ID NO: 31), wherein for (X₁₁) and (X₁₂) at least one applies: (X₁₁) is G, A, V, L, I, Y, H, K, R, Q, N, E, D, S or T, preferably N, D, E or Q, and (X₁₂) is G, A, V, L, I, Y, H, K, R, Q, N, E, D, S or T and preferably N, with the proviso that (X₁₁)-(X₁₂) are not D-G, D-S, N-G or N-S; or an amino acid sequence being at least 90%, preferably at least 95% identical thereto, provided that the three CDRs of the V_{H} region are determined by the amino acid sequences of GYAF(X₁)(X₂)(X₃)W(X₄)N(SEQ ID NO: 32), wherein for (X₁) to (X₃) at least one applies: (X₁) is G, A, V, L, I, Y, H, K, R, Q, N, E, D, S or T, preferably G, A, Y, H, K, R, Q, N, E, D, S or T, more preferably N or Q, and most preferably Q, (X₂) is P or T, and (X₃) is G, A, V, L, I, Y, H, K, R, Q, N, E, D, T or S and is preferably S, with the proviso that (X₁) to (X₃) are not N-(Z)-S/T, wherein (Z) can be any amino acid but not P, or wherein (X₁) to (X₃) are N-T-S; and (X₄) is M, I or L and preferably M, RIYPG(X₅)(X₆)(X₇)(X₈)NY(X₉)(X₁₀)KFK (SEQ ID NO: 33), wherein for (X₅) and (X₆) at least one applies: (X₅) is G, A, V, L, I, Y, H, Q, N, E, D, S or T and preferably D or E, and (X₆) is G, A, V, L, I, H, K, R, Q, N, E, D, S or T, preferably G or A, with the proviso that (X₅)-(X₆) are not D-S, N-G or N-S; for (X₇) and (X₈) at least one applies: (X₇) is G, A, V, L, I, Y, H, Q, N, E, D, S or T, preferably D, E or S, and (X₈) is G, A, V, L, H, Q, N, E, D, S or T and preferably T or A, with the proviso that (X₇)-(X₈) are not D-G, D-S, N-G or N-S; for (X₉) and (X₁₀) at least one applies: (X₉) is G, A, V, L, I, Y, H, K, R, Q, N, E, D, S or T and preferably N, E or Q, and (X₁₀) is G, A, V, L, I, Y, H, K, R, Q, N, E, D, S or T, preferably G or A, with the proviso that (X₉)-(X₁₀) are not D-G, D-S or N-S; and SEQ ID NO: 5, and the three CDRs of the V_{L} region are determined by the amino acid sequences of SASOGI(X₁₁)(X₁₂)FLT (SEQ ID NO: 34), wherein for (X₁₁) and (X₁₂) at least one applies: (X₁₁) is G, A, V, L, I, Y, H, K, R, Q, N, E, D, S or T, preferably N, D, E or Q, and (X₁₂) is G, A, V, L, I, Y, H, K, R, Q, N, E, D, S or T and preferably N, with the proviso that (X₁₁)-(X₁₂) are not D-G, D-S, N-G or N-S; SEQ ID NO: 7, and SEQ ID NO: 8.

With respect to the proviso that (X₁) to (X₃) are not N-(Z)-S/T, wherein (Z) can be any amino acid but not P, it noted that this proviso excludes an N-glycosylation site, which will be explained in more detail herein below. In the sequon N-(Z)-S/T the amino acid position (Z) corresponds to amino acid position (X₂) in SEQ ID NOs 30 and 32. Because (X₂) in SEQ ID NOs 30 and 32 can be T or P the proviso that (X₁) to (X₃) are not N-(Z)-S/T, wherein (Z) can be any amino acid but not P, excludes the amin acids N-T-S/T as (X₁) to (X₃) from SEQ ID NOs 30 and 32.

In accordance with a preferred embodiment the first aspect of the invention relates to an antibody binding to prostate-specific membrane antigen (PSMA), wherein the antibody comprises the V_{H} region determined by the amino acid sequence of SEQ ID NO: 1 or an amino acid sequence being at least 90%, preferably at least 95% identical thereto; and the V_{L} region determined by the amino acid sequence of SEQ ID NO: 2 or an amino acid sequence being at least 90%, preferably at least 95% identical thereto, provided that the three CDRs of the V_{H} region are determined by the amino acid sequences of SEQ ID NOs 3 to 5, and the three CDRs of the V_{L} region are determined by the amino acid sequences of SEQ ID NOs 6 to 8.

The term “antibody” as used in accordance with the present invention comprises monoclonal antibodies. Furthermore, also derivatives or fragments thereof, which still retain the binding specificity to the target, e.g. PSMA, are comprised in the term “antibody”. Antibody fragments or derivatives comprise, inter alia, Fab or Fab′ fragments, Fd, F(ab′)2, Fv or scFv fragments, chimeric antigen receptors (CARs) as well as multimeric formats such as minibodies, diabodies, tribodies or triplebodies, tetrabodies or chemically conjugated Fab′-multimers (see, for example, Harlow and Lane “Using Antibodies: A Laboratory Manual” Cold Spring Harbor Laboratory Press, 1999; Altshuler EP, Serebryanaya DV, Katrukha AG. 2010, Biochemistry (Mosc)., vol. 75(13), 1584; Holliger P, Hudson P J. 2005, Nat Biotechnol., vol. 23(9), 1126). The multimeric formats in particular comprise bispecific antibodies that can simultaneously bind to two different types of antigen. The first antigen can be found on PSMA. The second epitope may, for example, be another tumor marker that is specifically expressed on cancer cells or a certain type of cancer cells or immune cells, such as T-cells, monocytes/macrophages, or NK cells. Non-limiting examples of bispecific antibodies formats are Biconics (bispecific, full length human IgG antibodies), DART (Dual-affinity Re-targeting Antibody) and BiTE/BiKE (consisting of two single-chain variable fragments (scFvs) of different antibodies) molecules (Kontermann and Brinkmann (2015), Drug Discovery Today, 20(7):838-847).

The term “antibody” according to the invention is a humanized antibody; i.e. a human antibody with the exception of non-human CDRs of the antibody.

Various techniques for the production of antibodies are well known in the art. Examples for such techniques are described, e.g., in Harlow E and Lane D, Cold Spring Harbor Laboratory Press, 1988; Harlow E and Lane D, Using Antibodies: A Laboratory Manual, Cold Spring Harbor Laboratory Press, 1999. The parent 5D3 mAb is a murine antibody that was prepared by immunizing BALB/c mice with purified recombinant human PSMA. Furthermore, recombinant antibodies may be obtained from monoclonal antibodies or can be prepared de novo using various display methods such as phage, ribosomal, mRNA, or cell display. A suitable system for the expression of the recombinant (humanized) antibodies may be selected from, for example, bacteria, yeast, insects, mammalian cell lines or transgenic animals or plants (see, e.g., U.S. Pat. No. 6,080,560; Holliger P, Hudson P J. 2005, Nat Biotechnol., vol. 23(9), 11265). Further, techniques described for the production of single chain antibodies (see, inter alia, U.S. Pat. No. 4,946,778) can be adapted to produce single chain antibodies specific for an epitope of PSMA. Surface plasmon resonance as employed in the BIAcore system can be used to measure the affinity of antibodies.

The prostate-specific membrane antigen (PSMA) is also known as glutamate carboxypeptidase II (GCPII) or N-acetyl-L-aspartyl-L-glutamate peptidase I (NAALADase 1) or NAAG peptidase. PSMA is an enzyme that in humans is encoded by the FOLH1 (folate hydrolase 1) gene. Human PSMA comprises 750 amino acids and has a molecular weight of approximately 84 kDa. Human PSMA is highly expressed in the prostate. In some prostate cancers, PSMA is the second-most upregulated gene product, with an 8- to 12-fold increase over levels in noncancerous prostate cells. Because of this high expression, PSMA is used as biomarker for therapy and imaging of some cancers. In human prostate cancer, the higher expressing tumors are associated with quicker time to progression and a greater percentage of patients suffering from relapse.

The antibody of the invention specifically binds to prostate-specific membrane antigen (PSMA).

The amino acid sequences of SEQ ID Nos 3 to 5 are identical to the three CDRs of the V_{H} region (V_{H} CDR1, V_{H} CDR2 and V_{H} CDR3) of the murine monoclonal antibody 5D3 and SEQ ID Nos 6 to 8 (V_{L} CDR1, V_{L} CDR2 and V_{L} CDR3) are identical to the three CDRs of the V_{L} region of the murine monoclonal antibody 5D3. The corresponding nucleotide sequences of the six CDRs are shown in SEQ ID Nos 11 to 16.

SEQ ID NO: 32 is based on the V_{H} CDR1 of the of the murine monoclonal antibody 5D3 (SEQ ID NO: 3), wherein amino acids at positions X₁, X₂, X₃ and X₄ of SEQ ID NO: 32 list amino acids that do not change or essentially do not change the binding properties as compared to the corresponding amino acids at these positions in the V_{H} CDR1 of the of the murine monoclonal antibody 5D3 (SEQ ID NO: 3) and that may even confer technical advantages to the antibody of the invention that will be discussed in more detail herein below and that are illustrated by the examples. Similarly, SEQ ID NO: 33 is based on the V_{H} CDR2 of the of the murine monoclonal antibody 5D3 (SEQ ID NO: 4), wherein amino acids at positions X₅, X₆, X₇, X₈, X₉ and X₁₀ of SEQ ID NO: 33 list amino acids that do not change or essentially do not change the binding properties as compared to the corresponding amino acids at these positions in the V_{H} CDR2 of the of the murine monoclonal antibody 5D3 (SEQ ID NO: 4) and that may even confer technical advantages to the antibody of the invention that will be discussed in more detail herein below and that are illustrated by the examples.

SEQ ID NO: 34 is based on the V_{I} CDR1 of the of the murine monoclonal antibody 5D3 (SEQ ID NO: 6), wherein amino acids at positions X₁₁ and X₁₂ of SEQ ID NO: 34 list amino acids that do not or essentially do not change the binding properties as compared to corresponding amino acids at these positions in the V_{H} CDR1 of the of the murine monoclonal antibody 5D3 (SEQ ID NO: 6) and that may even confer technical advantages to the antibody of the invention that will be discussed in more detail herein below and that are illustrated by the examples.

The amino acid sequences of SEQ ID Nos 1 and 2 are the V_{H} region and the V_{L} region of the exemplified version 6 of a humanized 5D3 antibody (antibody 6; see examples section herein below) and the corresponding nucleotide sequences are SEQ ID Nos 9 and 10. SEQ ID Nos 1 and 2 comprise the set of the six CDRs of SEQ ID Nos 3 to 8 but the mouse constant regions and V framework regions of the 5D3 antibody were replaced by human sequences in antibody 6. Similarly, SEQ ID Nos 30 and 31 comprise the set of the six CDRs of SEQ ID Nos 32, 33, 6, 34, 7 and 8 but the mouse constant regions and V framework regions of the 5D3 antibody were replaced by human sequences in antibody 6.

In accordance with the first aspect of the invention amino acid sequences being at least 90%, preferably at least 95% identical to SEQ ID NO: 30 and SEQ ID NO: 31 are envisioned, provided that the six CDRs of SEQ ID Nos 32, 33, 6, 34, 7 and 8 remain unchanged. In accordance with the first aspect of the invention also amino acid sequences being at least 90%, preferably at least 95% identical to SEQ ID NO: 1 and SEQ ID NO: 2 are envisioned, provided that the six CDRs of SEQ ID Nos 3 to 8 remain unchanged. Since such amino acid sequences retain a high sequence identity with SEQ ID NO: 30 and SEQ ID NO: 31 or SEQ ID NO: 1 and SEQ ID NO: 2, respectively, it can safely be assumed that such antibodies retain the favorable properties of antibody 6 that will be discussed herein below.

In accordance with the present invention, the term “percent (%) sequence identity” describes the number of matches (“hits”) of identical nucleotides/amino acids of two or more aligned nucleic acid or amino acid sequences as compared to the number of nucleotides or amino acid residues making up the overall length of the template nucleic acid or amino acid sequences. In other terms, using an alignment for two or more sequences or subsequences the percentage of amino acid residues or nucleotides that are the same (e.g., 90% or 95% identity) may be determined, when the (sub)sequences are compared and aligned for maximum correspondence over a window of comparison, or over a designated region as measured using a sequence comparison algorithm as known in the art, or when manually aligned and visually inspected. This definition also applies to the complement of any sequence to be aligned.

Nucleotide and amino acid sequence analysis and alignment in connection with the present invention are preferably carried out using the NCBI BLAST algorithm (Stephen F. Altschul, Thomas L Madden, Alejandro A. Schaffer, Jinghui Zhang, Zheng Zhang, Webb Miller, and David J. Lipman (1997), Nucleic Acids Res. 25:3389-3402). BLAST can be used for nucleotide sequences (nucleotide BLAST) and amino acid sequences (protein BLAST). The skilled person is aware of additional suitable programs to align nucleic acid and amino acid sequences.

The antibody of the invention binds PSMA with increased preference with a Ko of 200 nM or lower, 100 nM or lower, 50 nM or lower, 10 nM or lower, 5 nM or lower, 3 nM or lower and 2 nM or lower. It is known in the art that the specific KD value also depends on the particular antibody format, e.g., Fab or full-length antibody; see examples herein below.

The term “K_{D}” refers to the equilibrium dissociation constant (the reciprocal of the equilibrium binding or affinity constant) and is used herein according to the definitions provided in the art.

The K_{D} value with which the antibody of the invention binds PSMA can be determined by well-known methods including, without being limiting, fluorescence titration, competition ELISA, calorimetric methods, such as isothermal titration calorimetry (ITC), flow cytometric titration analysis (FC titration), radioligand binding assays, biolayer interferometry (BLI), and surface plasmon resonance spectroscopy (BIAcore). Such methods are well known in the art and have been described e.g., in De Jong, L. A. A. et al. [2005]J. Chromatogr. B 829(1-2):1-25; Heinrich, L et al. [2010]J. Immunol. Methods 352(1-2):13-22.

Preferably, ELISA or competition ELISA, biolayer interferometry (BLI), or surface plasmon resonance (BIAcore) is employed to determine the K_{D}. Even more preferably, the K_{D} is determined by ELISA.

The antibody of the invention displays with increased preference a thermal stability as a purified protein of at least 64° C., at least 65° C., at least 66° C., and at least 67° C. The thermal stability is preferably determined by differential scanning fluorimetry (nanoDSF). NanoDSF measures the thermal denaturation temperature and, hence, the stability of a protein based on the intrinsic fluorescence of tryptophan residues of the protein.

Also, the antibody of the invention displays with increased preference an expression yield from transiently transfected suspension HEK 293 (Human Embryonic Kidney 293) T17 cells of at least 1.0 mg/250 mL, at least 1.25 mg/250 mL and at least 1.5 mg/250 mL Human embryonic kidney 293 cells, also often referred to as HEK cells, are a specific cell line originally derived from human embryonic kidney cells grown in tissue culture taken from an aborted female fetus. HEK 293 cells are used in the biotechnology industry to produce therapeutic or diagnostic proteins, such as antibodies.

It is shown in the appended examples that a number of different humanized 5D3 antibodies were produced on the basis of the three-dimensional structure of the 5D3 antibody and that antibody 6 was clearly the best humanized 5D3 antibody. Antibody 6, as full-length antibody as well as the Fab fragment, fully retained the nanomolar affinity (FIG. 4 and Table 1, for example, 1.8 nM in the Fab format of antibody 6 as determined by ELISA;) and single target specificity of the parent 5D3 full length antibody and the Fab fragment. On the other hand, antibody 5 (another humanized version of 5D3) reveals unspecific binding on PSMA-negative cell material. In addition, the production yield of the Fab fragment of antibody 6 (FIG. 1 and Table 1, 6.4 mg/L) was nearly as good as the production yield of the Fab of 5D3 antibody (FIG. 1 and Table 1, 10.8 mg/L), whereas the yield of the second-best humanized version of the 5D3 antibody was significantly lower (FIG. 1 and Table 1, antibody 2, only 0.6 mg/L). Yet further, antibody 6 (FIG. 3 and Table 1, for example, 67.5° C. in the Fab format) essentially retained the temperature stability of the 5D3 antibody (FIG. 3 and Table 1, for example, 66.4° C. in the Fab format). Antibody 6 is as good as the 5D3 antibody for therapeutic and diagnostic in vivo applications because antibody 6 retains the nanomolar affinity and the single target specificity the 5D3 antibody. At the same time antibody 6—in contrast to the murine antibody 5D3—is not expected to induce an adverse immune response when being administered to a human subject because of the replacement of the mouse framework regions by human framework regions. The high production yield is important for the commercial use of the antibody and the temperature stability for the in vivo stability within the body of a subject. The above discussion shows that antibody 6 performs outstandingly well as it is the only antibody that retained high expression yields, temperature stability and specificity of the parent 5D3 mAb. It was unexpected that a humanized variant of 5D3 murine antibody can be obtained which retains these three favorable characteristics of the parent 5D3 mAb despite the CDR-grafting during humanization.

It is furthermore shown in the appended examples that at amino acids positions 30, 31, 32, 34, 55, 56, 57 and 52 of the V_{H} chain of the antibody 6 as well as at amino acids positions 30 and 31 of the V_{L} chain of the antibody 6 amino acid can be substituted by selected other amino acids without diminishing or essentially without diminishing the outstanding performance of the antibody 6. Amino acids positions 30, 31, 32 and 34 of the V_{H} chain are within the V_{H} CDR1, amino acids positions 55, 56, 57 and 52 of the V_{H} chain are within the V_{H} CDR2, and amino acids positions 30 and 31 of the V_{L} chain are within the V_{L} CDR1. These amino acid positions correspond to the variable positions X₁ to X₁₂ in SEQ ID NOs 30 to 34.

For the development of antibody therapeutics is important that the lead antibody has a limited sequence liability load to increase chances of successful project completion and decrease the attrition rate. Therefore, in the examples in silico prediction was used to the assess liabilities of the CDRs of antibody 6. It was found that antibody 6 has a high sequence liability load, including a N-glycosylation motif in V_{H} CDR1 (N30-T31-S32), a high-risk aspartate isomerization site in V_{H} CDR2 (D55-G56), and a high-risk asparagine deamidation site in V_{H} CDR2 (N61-G62). For example, N-glycosylation in CDR is not desired for the development of a therapeutic antibody as it may have a strong impact on biological activity and cannot be adequately controlled during manufacture process. Furthermore, low risk sites were identified for methionine oxidation in V_{H} CDR1 (M34), aspartate isomerization in V_{H} CDR2 (D57-T58), and asparagine deamidation V_{L} CDR1 V_{L} (N30-N31). Based on the in-silico prediction selected antibody mutants were designed. These antibody mutants show that the mutations predicted by the in-silico analysis and combinations thereof are permissible. The antibody mutants display biophysical characteristics matching the parent antibody while at the same time they reduce the discussed liabilities. For instance, all antibody mutants retained a binding affinity of below 3 nM as determined by ELISA and a thermal stability of at least 64° C.

In view of the above findings on the liabilities of antibody 6 the present invention also relates to an antibody binding to prostate-specific membrane antigen (PSMA), wherein the three CDRs of the V_{H} region are determined by the amino acid sequences of GYAF(X₁)(X₂)(X₃)W(X₄)N(SEQ ID NO: 32), wherein for (X₁) to (X₃) at least one applies: (X₁) is G, A, V, L, I, Y, H, K, R, Q, N, E, D, S or T, preferably G, A, Y, H, K, R, Q, N, E, D, S or T, more preferably N or Q, and most preferably Q, (X₂) is P or T, and (X₃) is G, A, V, L, I, Y, H, K, R, Q, N, E, D, T or S and is preferably S, with the proviso that (X₁) to (X₃) are not N-(Z)-S/T, wherein (Z) can be any amino acid but not P, or wherein (X₁) to (X₃) is N-T-S; and (X₄) is M, I or L and preferably M, RIYPG(X₅)(X₆)(X₇)(X₈)NY(X₉)(X₁₀)KFK (SEQ ID NO: 33), wherein for (X₅) and (X₆) at least one applies: (X₅) is G, A, V, L, I, Y, H, Q, N, E, D, S or T and preferably D or E, and (X₆) is G, A, V, L, I, H, K, R, Q, N, E, D, S or T, preferably G or A, with the proviso that (X₅)-(X₆) are not D-S, N-G or N-S; for (X₇) and (X₈) at least one applies: (X₇) is G, A, V, L, I, Y, H, Q, N, E, D, S or T, preferably D, E or S, and (X₈) is G, A, V, L, H, Q, N, E, D, S or T and preferably T or A, with the proviso that (X₇)-(X₈) are not D-G, D-S, N-G or N-S; for (X₉) and (X₁₀) at least one applies: (X₉) is G, A, V, L, I, Y, H, K, R, Q, N, E, D, S or T and preferably N, E or Q, and (X₁₀) is G, A, V, L, I, Y, H, K, R, Q, N, E, D, S or T, preferably G or A, with the proviso that (X₉)-(X₁₀) are not D-G, D-S or N-S; and SEQ ID NO: 5, and the three CDRs of the V_{L} region are determined by the amino acid sequences of SASQGI(X₁₁)(X₁₂)FLT (SEQ ID NO: 34), wherein for (X₁₁) and (X₁₂) at least one applies: (X₁₁) is G, A, V, L, I, Y, H, K, R, Q, N, E, D, S or T, preferably N, D, E or Q, and (X₁₂) is G, A, V, L, I, Y, H, K, R, Q, N, E, D, S or T and preferably N, with the proviso that (X₁₁)-(X₁₂) are not D-G, D-S, N-G or N-S; SEQ ID NO: 7, and SEQ ID NO: 8, with the proviso that with increasing preference at least one, at least two, at least three, at least four, at least five or all of (X₁), (X₂), (X₃), (X₄), (X₅), (X₆), (X₇), (X₈), (X₉), (X₁₀), (X₁₁), (X₁₂) is/are not N, T, S, M, D, G, D, T, N, G, N and N, respectively. The proviso excludes the amino acids that can be found at these positions in antibody 6 thereby excluding antibody 6 from the scope of the embodiment.

The above provision is requires preferably with increasing preference at least one, at least two, at least three, at least four, at least five or all six of (X₁), (X₂), and (X₃) not being NTS, (X₄) not being M, (X₅) and (X₆) not being DG, (X₇) and (X₈) not being DT, (X₉) and (X₁₀) not being NG, and (X₁₁) and (X₁₂) not being NN. This preferred proviso excludes the amino acids that can be found at one or more of (X₁)/(X₂)/(X₃), (X₄), (X₅)/(X₆), (X₇)/(X₉), (X₉)/(X₁₀) and (X₁₁)/(X₁₂) of antibody 6. As discussed above the exclusion of one or more of these motives of one to three amino acids leads to an antibody having a limited sequence liability load which in turn increases the chances of successful project completion and decrease the attrition rate.

This antibody retains with increasing preference at least 90%, at least 95%, at least 98% identity with SEQ ID NOs 30 and 31 as defined in connection with the first aspect of the invention, with the proviso that with increasing preference at least one, at least two, at least three, at least four, at least five or all of (X₁), (X₂), (X₃), (X₄), (X₅), (X₆), (X₇), (X₉), (X₉), (X₁₀), (X₁₁), (X₁₂) are not N, T, S, M, D, G, D, T, N, G, N and N, respectively. Also, here the above preferred proviso preferably applies. This antibody more preferably comprises or consists of SEQ ID NOs 30 and 31 as defined in connection with the first aspect of the invention, with the proviso that with increasing preference at least one, at least two, at least three, at least four, at least five or all of (X₁), (X₂), (X₃), (X₄), (X₅), (X₆), (X₇), (X₈), (X₉), (X₁₀), (X₁₁), (X₁₂) are not N, T, S, M, D, G, D, T, N, G, N and N, respectively. Again, the above preferred proviso preferably applies. The preferred embodiments of the first aspect of the invention that will be described herein below apply mutatis mutandis to this antibody.

In accordance with a preferred embodiment of the first aspect of the invention the antibody comprises a V_{H} region differing by no more than 5, and preferably by no more than 3 amino acid substitutions from SEQ ID NO: 30, and/or a V_{L} region differing by no more than 5, and preferably by no more than 3 amino acid substitutions from SEQ ID NO: 31.

In accordance with a related preferred embodiment of the first aspect of the invention the antibody comprises a V_{H} region differing by no more than 5, and preferably by no more than 3 amino acid substitutions from SEQ ID NO: 1, and/or a V_{L} region differing by no more than 5, and preferably by no more than 3 amino acid substitutions from SEQ ID NO: 2.

In accordance with these preferred embodiments the framework regions within the V_{H} region and the V_{L} region of SEQ ID NO: 30 and SEQ ID NO: 31 or SEQ ID NO: 1 and SEQ ID NO: 2 may only differ from the exact sequences of SEQ ID NO: 30 and SEQ ID NO: 31 or SEQ ID NO.1 and SEQ ID NO: 2, each independently by no more than 5 or 3 amino acid substitutions. The no more 3 amino acid substitutions are preferably 2 amino acid substitutions and most preferably 1 amino acid substitution.

Since the antibody of this preferred embodiment only allows for very few amino acid replacements as compared to SEQ ID NO: 30 and SEQ ID NO: 31 or SEQ ID NO: 1 and SEQ ID NO: 2, it can safely be assumed that such antibodies retain the above-discussed favorable properties of antibody 6.

The very few amino acid replacements are preferably in one or more of amino acid positions 3, 5 and 119 of the V_{H} domain and are most preferably selected from AA3 Gln to Lys, AA5 Leu to Gln and AA19 Gly to Ser and/or are preferably in one or more of amino acid positions 3, 4 and 104 of the V_{L} domain and are most preferably selected from AA3 Gln to Glu, AA4 Met to Leu and AA04 Val to Leu.

These specific exchanges do not affect efficacy of the antibody. This was tested experimentally. Moreover, these positions are frequently modified in the process of cloning of antibody genes in expression constructs as published in Skerra, 1994. Gene, 141:79-84.

In accordance with a more preferred embodiment of the first aspect of the invention the amino acid substitutions are conservative amino acid substitutions.

It is well known that particular conservative amino acid substitutions in the framework regions of an antibody essentially do not affect the properties of the antibody.

The term “conservative amino acid substitution” designates the replacement of an amino acid by another amino acid having a side chain with similar biochemical properties. The naturally occurring amino acids can be classified as shown in the following table:

| Class | Amino acids | 1-letter code |
|---|---|---|
| Aliphatic | Alanine, Valine, Leucine, Isoleucine | A, V, L, I |
| S/Se-containing | Cysteine, Selenocysteine, Methionine | C, U, M |
| Cyclic | Proline | P |
| Non-chiral | Glycine | G |
| Aromatic | Phenylalanine, Tyrosine, Tryptophan | F, Y, W |
| Basic | Histidine, Lysine, Arginine | H, K, R |
| Acidic | Aspartate, Glutamate, | D, E |
| Hydrophilic | Serine, Threonine, Asparagine, Glutamine | S, T, N, Q |

It is preferred that a conservative amino acid substitution is the replacement of (i) an aliphatic amino acid (G, A, V, L, I) by another aliphatic amino acid, (ii) a S/Se-containing amino acid (C, U, M) by another S/Se-containing amino acid, (iii) an aromatic amino acid (F, Y, W) by another aromatic amino acid, (iv) a basic amino acid (H, K, R) by another basic amino acid, (v) an acidic amino acid (D, E) by another acidic amino acid, or (vi) a hydrophilic amino acid (S, T, N, Q) by another hydrophilic amino acid.

In accordance with a preferred embodiment of the first aspect of the invention the PSMA is determined by the amino acid sequence of SEQ ID NO: 29.

SEQ ID NO: 29 shows the amino acid sequence of the human PSMA protein. The human PSMA protein was used to generate the 5D3 antibody.

In accordance with a further preferred embodiment of the first aspect of the invention the antibody lacks an N-glycosylation site in the V_{H} region, with the proviso that (X₁) to (X₃) are not N-(Z)-S/T, wherein (Z) can be any amino acid but not P.

It is to be understood that this preferred embodiment ensures that the V_{H} region and in particular the V_{H} CDR1 of the antibody of the invention does not comprise a N-glycosylation site. Hence, the above preferred embodiment ensures that the antibody of the invention does not comprise a N-glycosylation site in the amino acid sequence of the V_{H} region.

Protein N-glycosylation is a widespread posttranslational modification where an oligosaccharide chain is attached to the nitrogen atom of the side chain amide group of asparagine that is a part of the N-X-S/T glycosylation sequon, where X≠P. This posttranslational modification is important for both the structure and function of many proteins. Numerous therapeutic proteins are N-linked glycoproteins and the importance of N-linked glycosylation is becoming increasingly evident for pharmaceuticals. First, N-linked oligosaccharides in proteins produced in non-human mammalian expression systems, e.g., NS0 or CHO cells, can differ from glycans produced in humans and may cause immunogenic reactions in treated subjects (patients). Additionally, N-linked oligosaccharides result from enzymatic posttranslational modification and are inherently heterogenous, which may also lead to an undesired heterogeneity in the preparation of a given pharmaceutical. It can be therefore advantageous to remove N-glycosylation sites in pharmaceuticals by known techniques, such as site-directed mutagenesis of the N-glycosylation sequon. This may be achieved either by directly replacing the glycosylated Asn side chain, for example via mutagenesis to Gln, Asp or Glu, or by modifying the other two positions of the sequon (N-X-S/T), for example by substituting residue X by Pro or by substituting the S/T position (i.e. position (X₃) of the antibody of the invention) with an amino acid residue different from Ser and Thr.

However, at the same time it is critical to preserve the functional properties of said pharmaceutical such as target affinity/specificity and stability.

In the case of antibodies, an N-glycosylation site is typically found in the Fc portion, where it also plays a role for the interaction of the immunoglobulin with Fc receptors on immune cells. In addition, some antibodies carry an N-glycosylation site in one of the variable domains. This extra N-glycosylation is often not relevant for antigen binding and, in contrast to the N-glycosylation of the Fc portion, it is not involved in the immunological effector function of the antibody. Thus, the elimination of an N-glycosylation site in the variable domain can be beneficial with regard to manufacturing or biotechnological processing of this biopharmaceutical, its stability and/or biological drug homogeneity. Consequently, a preferred embodiment of the antibody of the present invention lacks a functional N-glycosylation sequon in the V_{H} region and in particular in the first variable domain of the heavy chain (V_{H}).

In accordance with a further preferred embodiment of the first aspect of the invention the antibody is coupled to (a) a labelling group, (b) a toxin, (c) a drug, (d) a radionucleotide, (e) a photosensitizing functional group, (f) a cytokine, (g) a chemokine, (h) an enzyme, (i) a component modulating serum half-life, j) an antibody or an Fc part thereof, or (k) an antibody mimetic.

The labelling group is a group that allows the detection of the antibody, preferably in vivo in a subject. The labelling group is preferably a fluorescent dye. The fluorescent dye is preferably a component selected from Alexa Fluor dyes, (BODIPY) dye, Cy dyes, Dy dyes, IRDye dyes, HiLyte Fluor dyes, Oregon dyes, TRITC, Rhodamine, and Fluorescein. and derivatives thereof including, but not limited to, NHS esters, maleimides, phosphines, and free acids. Non-limiting further examples of fluorescent proteins are green fluorescent protein (GFP), yellow fluorescent protein (YFP), red fluorescent protein (RFP), cyan fluorescent protein (CFP) and infrared fluorescent protein (IFP). The labelling group is also preferably a contrast agent. A contrast agent as used herein is a substance used to enhance the contrast of structures or fluids within the body in medical imaging. Common contrast agents work based on X-ray attenuation or magnetic resonance signal enhancement.

The toxin is preferably a small organic compound or a polypeptide, more preferably a toxic compound selected from the group consisting of, but not limited to, calicheamicin, maytansinoid, neocarzinostatin, esperamicin, dynemicin, kedarcidin, maduropeptin, doxorubicin, daunorubicin, auristatin, Ricin-A chain, modeccin, truncated Pseudomonas exotoxin A, diphtheria toxin and gelonin.

The drug is a compound that is capable to treat or prevent a disease. The disease is preferably a tumor and most preferably prostate cancer. The drug is preferably 2-PMPA or analogue thereof, thiol or an indole thiol derivative, a hydroxamate derivative, a conformationally constricted dipeptide mimetic or a PBDA- and urea-based inhibitor. These drugs are PSMA inhibitors that hold promise in therapy and diagnosis (Zhou et al. (2005). Nature Reviews. Drug Discovery. 4 (12): 1015-26).

The radionuclide is preferably either selected from the group of gamma-emitting isotopes, more preferably ^{99m}Tc, ¹²³I, ¹²⁵I, or ¹¹¹In, and/or from the group of positron emitters, more preferably ¹⁸F, ⁶⁰Cu, ⁶²Cu, ⁶⁴Cu, ⁶⁸Ga, ⁸⁶Y, ⁸⁹Zr, or ¹²⁴I, and/or from the group of beta-emitters, more preferably ¹³¹I, ⁹⁰Y, ¹⁷⁷Lu, or ⁶⁷Cu, or from the group of alpha-emitters, preferably ²¹³Bi, ²¹²Bi, ²²⁷Th, ²¹²Ph, ²²³Ra, ²²⁵Ac, or ²¹¹At. The radionuclide is more preferably a positron emitter since they are particularly suitable for diagnostics, e.g., via positron emission tomography imaging or single-photon emission computed tomography (SPECT). For therapeutic applications the radionuclide is more preferably an alpha-emitter or a beta-emitter.

Antibody can be used for photodynamic therapy by the attachment of suitable photosensitizing functional group comprising, but not limited to, porphyrins, chlorins, bacteriochlorins, phthalocyanines (e.g., IR700DX), phenothiazinium salts, benzophenothiazinium salts, squaraine, phenalenone, BODIPY dyes, ruthenium, rhodium, and iridium complexes, Hypericin, flavins, and genetically encoded proteins such as KillerRed protein.

The cytokine is preferably selected from the group consisting of IL-2, IL-12, TNF-alpha, IFN alpha, IFN beta, IFN gamma, IL-10, IL-15, IL-24, GM-CSF, IL-3, IL-4, IL-5, IL-6, IL-7, IL-9, IL-11, IL-13, LIF, CD80, B70, TNF beta, LT-beta, CD-40 ligand, Fas-ligand, TGF-beta, IL-1alpha and IL-1beta. As it is well known in the art, cytokines may favour a pro-inflammatory or an anti-inflammatory response of the immune system. Thus, depending on the disease to be treated either fusion constructs with a pro-inflammatory or an anti-inflammatory cytokine may be favoured. For example, for the treatment of cancer, in particular prostate cancer in general fusion constructs comprising pro-inflammatory cytokines are preferred. Anti-inflammatory cytokines may be used, for example, to avoid autoimmune reaction.

The chemokine is preferably selected from the group consisting of IL-8, GRO alpha, GRO beta, GRO gamma, ENA-78, LDGF-PBP, GCP-2, PF4, Mig, IP-10, SDF-1alpha/beta, BUNZO/STRC33, I-TAC, BLC/BCA-1, MIP-1alpha, MIP-1 beta, MDC, TECK, TARC, RANTES, HCC-1, HCC-4, DC-CK1, MIP-3 alpha, MIP-3 beta, MCP-1-5, eotaxin, Eotaxin-2, 1-309, MPIF-1, 6Ckine, CTACK, MEC, lymphotactin and fractalkine.

An enzyme is a protein that catalyzes a particular chemical or biochemical reaction. Antibody-enzyme fusion proteins have been used, for example, to target tumors for cancer therapy in two ways. In one system, an antibody-enzyme is pretargeted to the tumor followed by administration of an inactive prodrug that is converted to its active form by the pretargeted enzyme. This system has been described as antibody-directed enzyme prodrug therapy (ADEPT). Suitable enzymes for prodrug activation will be further discussed herein below in the section on enzymes. The other system uses antibody-enzyme fusion proteins as direct therapeutics, where the enzyme is toxic by itself. The key feature in this approach is that the antibody is used to target and subsequently internalize the toxic enzyme into the tumor cell, which activates cell-death processes. This antibody-enzyme system has been largely applied to deliver ribonucleases.

Enzymes may also be used for imaging in diagnostics. Conjugation partners in this regard include enzymes capable of catalyzing chromogenic, chemiluminescent or fluorescent reactions, such as e.g., horseradish peroxidase (HRP), luciferase, alpha-galactosidase and alkaline phosphatase (AP). For example, the conjugation partner can also be an enzyme capable of liberating or activating cytotoxic agents that have been brought into the vicinity of the targeted tissue, for example an enzyme for prodrug activation, such as e.g., an enzyme selected from the group consisting of carboxy-peptidases, glucuronidases and glucosidases (Bagshawe, K. D. [2009] Curr. Drug Targets 10:152-157; Chen, K.-C. [2011] Bioconjugate Chem. 22:938-948.). For certain applications a truncated version of an enzymes is preferred, for example by omitting a binding domain, provided that the truncated version retains or essentially retains the enzymatic activity of the full-length enzyme. Thus, with respect to the truncated version of the enzymes it is to be understood that they retain or essentially retain the enzymatic activity of the full-length enzyme.

Several compounds are available to extend the half-life of therapeutics, including antibodies. Two of the most common and preferred options are PEGylation and fusion with human serum albumin. Other options are the chemical coupling of polymers and carbohydrates, post-translational modifications such as N-glycosylation, and fusion to recombinant polymer mimetics.

The antibody to be coupled to the antibody of the invention is generally distinct from the antibody of the invention. By the second antibody a second binding specificity to an antigen other than PSMA can be coupled to the anti-PSMA antibody of the invention, thereby generating a bispecific construct. The coupling of an Fc part of an antibody to the antibody of the invention is a further means for extending the in vivo half-life of the antibody of the invention.

As used herein, the term “antibody mimetics” refers to compounds or proteins which, like antibodies, can specifically bind antigens, but which are not structurally related to antibodies. Antibody mimetics are usually artificial peptides or proteins with a molar mass of about 3 to 30 kDa. The antibody mimetic is preferably selected from the group consisting of an Anticalin, Affibody, Adnectin, DARPin, Avimer, Nanofitin, Affilin, β-Wrapin, ADAPT, Monobody, Rasln, FingR, Pronectin, Centyrin, Affilin, Affimer, Adhiron, Affitin, aRep, Repebody, i-body, Fynomer or Kunitz domain protein.

“Anticalins” will be discussed herein below.

“Affibodies”, in accordance with the present invention, are a family of antibody mimetics derived from the Z-domain of staphylococcal protein A. Affibodies are structurally based on a three-helix bundle domain. An affibody has a molecular mass of around 6 kDa and is stable at high temperatures and under acidic or alkaline conditions. Target specificity is obtained by randomisation of amino acids located in two alpha-helices involved in the binding activity of the parent protein domain (Feldwisch, J & Tolmachev, V. [2012] Methods Mol. Biol. 899:103-126).

“Adnectins” and also “Monobodies”, in accordance with the present invention, are based on the 10th extracellular domain of human fibronectin Ill (10Fn3), which adopts an Ig-like sandwich fold with 2 to 3 exposed loops, but lacks the central disulphide bridge (Gebauer, M. & Skerra, A. [2009] Curr. Opin. Chem. Biol. 13:245-255 and Koide et al. 1998, J. Mol. Biol. 284:1141-51). Adnectins and Monobodies with the desired target specificity can be genetically engineered by introducing modifications into specific loops or other surface areas of the protein.

“DARPins”, in accordance with the present invention, are designed ankyrin repeat domains that provide a rigid interface arising from typically three repeats corresponding to an artificial consensus sequence, whereby six positions per repeat are randomised. Consequently, DARPins lack structural flexibility (Gebauer, M. & Skerra, A. [2009] Curr. Opin. Chem. Biol. 13:245-255).

The term “Avimer”, as used herein, refers to a class of antibody mimetics which consist of two or more peptide sequences of 30 to 35 amino acids each, which are derived from A-domains of various membrane receptors, and which are connected by linker peptides. Binding of target molecules occurs via the A-domain and domains with desired binding specificity can be selected, for example, by phage display techniques. The target specificity of the different A-domains contained in an avimer may, but do not have to be identical (Weidle UH, et al., (2013), Cancer Genomics Proteomics; 10(4):155-68).

“Nanofitins” and also an “Affitins” are antibody mimetic proteins that are derived from the DNA binding protein Sac7d of Sulfolobus acidocaldarius. Nanofitins and Affitins usually have a molecular weight of around 7 kDa and are designed to specifically bind a target molecule by randomising the amino acids on the binding surface (Mouratou B, Behar G, Paillard-Laurance L, Colinet S, Pecorari F., (2012) Methods Mol Biol.; 805:315-31.

The term “Affilin”, as used herein, refers to antibody mimetics that are developed by using either gamma-B crystalline or ubiquitin as a scaffold and modifying amino acids on the surface of these proteins by random mutagenesis. Selection of affilins with the desired target specificity is achieved, for example, by phage display or ribosome display techniques. Depending on the scaffold, affilins have a molecular weight of approximately 10 or 20 kDa. As used herein, the term affilin also refers to di- or multimerized forms of affilins (Weidle UH, et al., (2013), Cancer Genomics Proteomics; 10(4):155-68).

As used herein, the term “β-Wrapins” designates affibody protein homodimers with a disulfide bond between the pair of Cys28 residues connecting the two identical monomer subunits, referred to as subunits 1 and 2. The scaffold used in engineering β-wrapins is ZAβ₃, an Aβ-binding affibody protein that not only prohibits the initial aggregation of Aβ monomers into toxic forms, but also dissociates pre-formed oligomeric aggregates by sequestering and stabilizing a β-hairpin conformation of Aβ monomers (Orr et al. (2018), Computers & Chemical Engineering, 116(4):322-332).

As used herein, the term “ABD-Derived Affinity Proteins (ADAPT)” refers to a class of antibody mimetics that has been created using the albumin-binding domain (ABD) of streptococcal protein G as a stable protein scaffold (Garousi et al (2015), Cancer Res.; 75(20):4364-71). By diversifying a surface of the domain that is not directly involved in albumin binding, molecules can be selected to bind a novel target and still retain their ability to bind albumin. This strategy has been used to select binders to a number of proteins, for example, the cancer-related epidermal growth factor receptor 3.

As used herein “Rasins” are 10FnIII-based antibody mimetics. Hence, they use the 10<h domain of human fibronectin as their scaffold. RasIns are disulfide-free intrabodies. They were shown to be stable inside cells and also when fused with a fluorescent protein label (Cetin eat al. (2017), J Mol Biol.; 429(4):562-573).

As used herein, the term “FingRs (Fibronectin intrabodies generated with mRNA display)” designates recombinant antibody-Ike proteins also being based on the 10Fnll scaffold (Gross eat al. (2013), Neuron.; 78(6): 971-985.).

As used herein, the term “Pronectins” designates recombinant antibody-Ike proteins being based on the fourteenth type-Ill scaffold of human fibronectin (14Fn3). The well-characterized fibronectin protein is prevalent throughout the human body. Human fibronectin, an extracellular protein, is naturally abundant in human serum. Intelligent loop-diversity has been designed to closely mimic the natural human repertoire and avoid sequence immunogenicity. The intrinsic properties of a Pronectin align with the pharmacological properties needed to make it a successful drug, including high potency, specificity, stability, favorable small size, and high-yield production in E. coli and yeast (http://www.protelica.com/pronectintech.html).

As used herein, the term “Centyrins” designates recombinant antibody-Ike proteins being based on the consensus tenascin FN3 framework (Tencon) (Diem et al. (2014), Protein Eng., Des. And Sel. 27, 419-429). Centryins against different targets, e.g., human c-MET, rTNFα and mlL-17A, were generated.

As used herein, “Affimers” refer to small proteins that bind to target molecules with similar specificity and affinity to that of antibodies. These engineered non-antibody binding proteins are designed to mimic the molecular recognition characteristics of monoclonal antibodies in different applications. In addition, these affinity reagents have been optimized to increase their stability, make them tolerant to a range of temperatures and pH, reduce their size, and to increase their expression in E. coli and mammalian cells. Derived from the cysteine protease inhibitor family of cystatins, which function in nature as cysteine protease inhibitors, these 12-14 kDa proteins share the common tertiary structure of an α-helix lying on top of an anti-parallel β-sheet (Tiede et al. (2017), eLife.; 6: e24903).

The class of recombinant antibody-like proteins designated as “Adhirons” herein is based on a phytocystatin consensus sequence as the scaffold (Tiede et al. (2014) Protein Eng. Des. Sel. 27, 145-55).

The class of recombinant antibody-like proteins designated as “αRep” herein is derived from alpha-helicoidal HEAT-Ike repeat protein scaffolds. In more detail, the αRep proteins are derived from a natural family of modular proteins comprising alpha-helical repeats, related to HEAT repeats, named after Huntingtin, the elongation factor 3 (EF3), the protein phosphatase 2A (PP2A), and the yeast kinase TOR. The association of several HEAT repeats forms alpha-solenoids of various lengths, which are naturally found in a number of cellular proteins involved in intracellular transport and protein-protein interaction (Hadpech et al. (2017), Scientific Reports; 7: Article number16335).

As used herein, the term “Repebodies” designates recombinant antibody-like proteins which are composed of leucine-rich repeat (LRR) modules. In more detail, the binding scaffold of Repebodies is based on variable lymphocyte receptors, which are nonimmunoglobulin antibodies composed of LRR modules in jawless vertebrates. A template scaffold was first constructed by joining consensus repeat modules between the N- and C-capping motifs of variable lymphocyte receptors. The N-terminal domain of the template scaffold was redesigned based on the intemalin-B cap by analyzing the modular similarity between the respective repeat units using a computational approach (Lee at al. (2012), Proc Natl Acad Sci; 109(9): 3299-3304).

As used herein, the term “i-bodies” refers to recombinant antibody-like proteins built on the scaffold of a human protein and engineered with two loops that mimic the shape of shark antibodies. These loops are responsible for binding or interacting with a particular target (in circulation or on a cell) that is causing disease. The i-body is a human analogue of the antigen binding domain of the shark antibody, which combines the advantages of monoclonal antibodies (high target specificity and affinity) with the beneficial stability features of small molecules (httpsJ/www.ibodies.eu/).

As used herein, the term “Fynomer” refers to a non-immunoglobulin-derived binding polypeptide derived from the human Fyn SH3 domain. Fyn SH3-derived polypeptides are well-known in the art and have been described e.g., in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDa and domains with the required target specificity can be selected by display techniques such as phage display (Weidle et al., (2013), Cancer Genomics Proteomics; 10(4):155-68).

In accordance with a more preferred embodiment of the first aspect of the invention the antibody mimetic is an anticalin.

Lipocalin-derived binding proteins, also referred to as anticalins, represent a class of non-immunoglobulin binding proteins based on the human lipocalin scaffold. Lipocalins comprise a diverse family of small (20 kDa) extracellular proteins that occur in many species ranging from bacteria to humans and serve for the transport or scavenging of physiological compounds. Despite mutually low sequence homology, the three-dimensional fold of lipocalins is highly conserved (Rothe and Skerra (2018), BioDrugs. 2018; 32(3): 233-243.). Anticalins constitute an emerging class of artificial binding proteins obtained by combinatorial design based on the compact and robust human lipocalin scaffold. Due to their human origin, anticalins have low immunogenic potential, and in several clinical trials anticalins with different target specificities have demonstrated safety.

The single chain molecular architecture of lipocalin-derived binding proteins is dominated by a compact eight-stranded anti-parallel β-barrel. At the open end of the barrel there are four loops connecting each pair of β-strands. The four structurally variable loops are referred to herein as “loop regions”, whereas the remainder of the protein makes up the framework or “frame regions”. Thus, similar to the structure of antibodies, the lipocalin-derived binding proteins as used in the present invention are essentially made of conserved frame(work) regions that are generally not directly involved in the binding to the exogenous ligand, as well as hypervariable, specificity-determining segments with amino acid residues being involved in the binding to the exogenous ligand (here the loop regions, which might be seen as resembling the CDRs in antibodies).

Hence, lipocalin and also lipocalin-derived binding proteins consist of frame regions and loop regions according to the following scheme:
Frame 1-Loop 1-Frame 2-Loop 2-Frame 3-Loop 3-Frame 4-Loop 4-Frame 5

The lipocalin-derived binding protein is preferably a lipocalin 2 (Lcn2)-derived binding protein. Lipocalin-2 (Lcn2), also known as neutrophil gelatinase-associated lipocalin (NGAL), is a protein that in humans is encoded by the LCN2 gene. Human LCN2 mRNA is, for example, represented by the NCBI Reference Sequence: NM_005564.5 (as available on Mar. 12, 2019) and the amino acid sequence of human Lcn2 protein including the signal peptide is, for example, represented by the UniProt ID P80188-2 (as available on Nov. 1, 1995).

The present invention relates in a second aspect to a nucleic acid molecule encoding the antibody according to the first aspect of the invention.

In a related third aspect the invention relates to a set of two nucleic acid molecules, wherein the first nucleic acid molecule encodes the V_{H} region of the antibody according to the first aspect of the invention and the second nucleic acid molecule encodes the V_{L} region of the antibody according to the first aspect of the invention.

As far as the nucleic acid molecule(s) refer to an antibody being coupled to one of the compounds as defined in the above-items (a) to (k) it is to be understood that these compounds can also be encoded by the nucleic acid molecule(s) as long as the compound is a proteinaceous compound.

The term “nucleic acid molecule” in accordance with the present invention includes DNA, such as cDNA or double or single stranded genomic DNA and RNA. In this regard, “DNA” (deoxyribonucleic acid) means any chain or sequence of the chemical building blocks adenine (A), guanine (G), cytosine (C) and thymine (T), called nucleotide bases, that are linked together on a deoxyribose sugar backbone. DNA can have one strand of nucleotide bases, or two complementary strands which may form a double helix structure. “RNA” (ribonucleic acid) means any chain or sequence of the chemical building blocks adenine (A), guanine (G), cytosine (C) and uracil (U), called nucleotide bases, that are linked together on a ribose sugar backbone. RNA typically has one strand of nucleotide bases, such as mRNA. Included are also single- and double-stranded hybrids molecules, i.e., DNA-DNA, DNA-RNA and RNA-RNA. The nucleic acid molecule may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acid molecules, in the following also referred as polynucleotides, may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8: 1). LNA is an RNA derivative in which the ribose ring is constrained by a methylene linkage between the 2′-oxygen and the 4′-carbon. Also included are nucleic acids containing modified bases, for example thio-uracil, thio-guanine and fluoro-uracil. A nucleic acid molecule typically carries genetic information, including the information used by cellular machinery to make proteins and/or polypeptides. The nucleic acid molecule of the invention may comprise promoters, enhancers, response elements, signal sequences, polyadenylation sequences, introns, 5′- and 3′-non-coding regions, and the like.

The nucleic acid molecule according to the invention encodes the antibody of the invention. The antibody of the invention may also be encoded by a set of two nucleic acid molecules. This is because an antibody (a full-length antibody or fragments, such as scFv or Fab) comprises heavy and light chain sequences which, for example, upon expression in a cell, self-assemble into an antibody. The heavy and light chain sequences can be encoded by a set of two different nucleic acid molecules.

The present invention relates in a fourth aspect to a vector comprising the nucleic acid molecule or the set of two nucleic acid molecules of the invention in an expressible form, and optionally a proteinaceous compound as defined in any one of items (a) to (c) and (f) to (k) in an expressible form.

In a related fifth aspect the present invention relates to a set of two vectors, wherein the first vector comprises the first nucleic acid molecule of the invention in an expressible form and the second vector comprises the second nucleic acid molecule of the invention in an expressible form, and wherein the first and/or second vector optionally comprises a proteinaceous compound a defined in any one of items (a) to (c) and (f) to (k) in an expressible form.

The term “vector” in accordance with the invention means preferably a plasmid, cosmid, virus, bacteriophage or another vector used e.g., conventionally in genetic engineering which encoding the antibody of the invention in expressible from. For the same reasons as discussed in connection with the set of nucleic acid molecules of the invention, the antibody of the invention may also be encoded by a set of vectors, preferably by a set of two vectors.

The nucleic acid molecule(s) encoding the antibody of the invention may, for example, be inserted into several commercially available vectors. Non-limiting examples include prokaryotic plasmid vectors, such as of the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, plZD35, pLXIN, pSIR (Clontech), pIRES-EGFP (Clontech), pEAK-10 (Edge Biosystems) pTriEx-Hygro (Novagen) and pCINeo (Promega). Examples for plasmid vectors suitable for Pichia pastoris comprise e.g., the plasmids pAO815, pPIC9K and pPIC3.5K (all Invitrogen).

The nucleic acid molecules inserted into the vector can e.g., be synthesized by standard methods or isolated from natural sources. Ligation of the coding sequences to transcriptional regulatory elements and/or to other amino acid encoding sequences can also be carried out using established methods. Transcriptional regulatory elements (parts of an expression cassette) ensuring expression in prokaryotes or eukaryotic cells are well known to those skilled in the art. These elements comprise regulatory sequences ensuring the initiation of transcription (e. g., translation initiation codon, promoters, such as naturally-associated or heterologous promoters and/or insulators; see above), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers. Preferably, the polynucleotide(s) encoding the antibody of the invention is operatively linked to such expression control sequences allowing expression in prokaryotes or eukaryotic cells. The vector may further comprise nucleic acid sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the polynucleotide of the invention. Such leader sequences are well known in the art.

Furthermore, it is preferred that the vector comprises a selectable marker. Examples of selectable markers include genes encoding resistance to neomycin, ampicillin, hygromycin, and kanamycin. Specifically designed vectors allow the shuttling of DNA between different hosts, such as bacteria-fungal cells or bacteria-animal cells (e. g. the Gateway system available at Invitrogen). An expression vector according to this invention is capable of directing the replication, and the expression, of the polynucleotide and an encoded peptide or a fusion protein of this invention. Apart from introduction via vectors such as phage vectors or viral vectors (e.g., adenoviral, retroviral), the nucleic acid molecules as described herein above may be designed for direct introduction or for introduction via liposomes into a cell. Additionally, baculoviral systems or systems based on vaccinia virus or Semliki Forest virus can be used as eukaryotic expression systems for the nucleic acid molecules of the invention.

The present invention relates in a sixth aspect to a non-human host comprising nucleic acid molecule, the set of two nucleic acid molecules, the vector or the set of two vectors of the invention.

The term “host cell” means any cell of any organism that is selected, modified, transformed, grown, or used or manipulated in any way, for the production of the antibody of the invention by the cell and optionally a proteinaceous compound as defined in any one of items (a) to (c) and (f) to (k). The host cell is therefore generally an ex vivo or in vitro cell and/or an isolated cell.

The host cell of the invention is typically produced by introducing the nucleic acid molecule(s) or vector(s) of the invention into the host cell which upon its/their presence mediates the expression of the nucleic acid molecule(s) of the invention encoding the antibody of invention. The host from which the host cell is derived or isolated may be any prokaryote or eukaryotic cell or organism, preferably with the exception of human embryonic stem cells that have been derived directly by destruction of a human embryo.

Suitable prokaryotes (bacteria) useful as hosts for the invention are, for example, those generally used for cloning and/or expression like E. coli(e.g., E coli strains BL21, HB101, DH5a, XL1 Blue, Y1090 and JM101), Salmonella typhimurium, Serrada marcescens, Burkholderia glumae, Pseudomonas putida, Pseudomonas fluorescens, Pseudomonas stutzen, Streptomyces lividans, Lactococcus lactis, Mycobacterium smegmatis, Streptomyces coelicolor or Bacillus subtilis. Appropriate culture mediums and conditions for the above-described host cells are well known in the art.

A suitable eukaryotic host cell may be a vertebrate cell, an insect cell, a fungal/yeast cell, a nematode cell or a plant cell. The fungal/yeast cell may be a Saccharomyces cerevisiae cell, Pichia pastoris cell, Kluyveromyces lactis cell, or an Aspergillus cell. Preferred examples of a host cell to be genetically engineered with the nucleic acid molecule or the vector(s) of the invention is a cell of yeast, E. coli and/or a species of the genus Bacillus (e.g., B. subtilis). In one preferred embodiment the host cell is a yeast cell (e.g., S. cerevisiae, K. lactis, or P. pastors).

In a different preferred embodiment the host cell is a mammalian host cell, such as a Chinese Hamster Ovary (CHO) cell, mouse myeloma lymphoblastoid, human embryonic kidney cell (HEK-293), human embryonic retinal cell (Crucelrs Per.C6), or human amniocyte cell (Glycotope and CEVEC). The cells are frequently used in the art to produce recombinant proteins. CHO cells are the most commonly used mammalian host cells for industrial production of recombinant protein therapeutics for humans.

The present invention also relates to a transgenic animal, preferably a non-human transgenic animal comprising the vector or the set of two vectors of the invention.

Transgenic animals can be used for the production of antibodies as is reviewed, for example, in Bruggemann (2014), Arch Immunol Ther Exp (Warsz). 2015; 63(2): 101-108. The Transgenic animals is preferably a mammal other than human. The antibodies may also be produced such that the antibodies can be obtained from the milk of transgenic mammals. The mammal is therefore preferably a goat, sheep or cow.

The present invention relates in a seventh aspect to method for producing an antibody of the invention, comprising (a) culturing the host of the invention under conditions that allow synthesis of said antibody; and (b) recovering said antibody from said culture.

When in the following the term “antibody” is used in connection with the discussion of the various embodiments of the invention, it is to be understood that this optionally also includes the proteinaceous compound as defined in any one of items (a) to (c) and (f) to (k) and, where applicable, any non-proteinaceous compound as defined in any one of items (a) to (k).

The term “culturing” specifies the process by which host cells are grown under controlled conditions. These conditions may vary dependent on the host cell used. The skilled person is well-aware of methods for establishing optimized culturing conditions. Moreover, methods for establishing, maintaining and manipulating a cell culture have been extensively described in the state of the art.

Methods of isolation of the antibody of the invention are well-known in the art and comprise without limitation method steps such as ion exchange chromatography, gel filtration chromatography (size exclusion chromatography), affinity chromatography, high pressure liquid chromatography (HPLC), reversed phase HPLC, disc gel electrophoresis or immunoprecipitation, see, for example, Antibody Purification Handbook, GE Healthcare, 18-1037-46.

The term “recovering said antibody from said culture” in accordance with the invention refers to the product of a process implying that in the host cell a process can be induced by which information from nucleic acid molecule(s) encoding the antibody of the invention is/are used in the synthesis of the antibody of the invention. Several steps in this process may be modulated, including the transcription, RNA splicing, translation, and post-translational modification of the antibody of the invention by methods know in the art. Accordingly, such modulation may allow for control of the timing, location, and amount of antibody produced.

The present invention relates in an eighth aspect to a diagnostic composition or a pharmaceutical composition comprising the antibody, the nucleic acid molecule, the set of two nucleic acid molecules, the vector, the set of two vectors or the host cell of the invention.

In accordance with the present invention, the term “pharmaceutical composition” relates to compositions for administration to a subject, preferably a human subject. In connection with the pharmaceutical composition the subject may be a patient, i.e., subject having a disease. In accordance with the present invention, also a “diagnostic composition” may relate to a composition for administration to a subject, preferably a human subject. A “diagnostic composition” may furthermore relate to a composition to be contacted in vitro or ex vivo with a sample from a subject, preferably a human subject.

The diagnostic or pharmaceutical composition of the invention comprises the compounds recited above. It may, optionally, comprise further molecules capable of altering the characteristics of the compounds of the invention thereby, for example, stabilizing, modulating and/or activating their function. The composition may be in solid or liquid form or any other appropriate form and may be, inter alia, in the form of (a) powder(s), (a) tablet(s), (a) solution(s) or (an) aerosol(s). The composition may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents including DMSO etc. Compositions comprising such carriers can be formulated by well-known conventional methods.

While the diagnostic composition is to diagnose a disease within a subject or ex vivo or in vitro within on a sample from the subject, and in this regard, e.g., the presence, location and/or severity of the disease, the pharmaceutical composition is to treat or prevent the development of a disease in a subject.

The pharmaceutical compositions can be administered to the subject at a suitable dose in order to achieve a curative or disease preventive effect. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 1 to 50 mg antibody/kg bodyweight every 1, 2, 3 or 4 weeks. However, a more preferred dosage might be in the range of 2 to 25 mg/kg every 1, 2, 3 or 4 weeks, even more preferably 5 to 20 mg/kg every 1, 2, 3 or 4 weeks. The length of treatment needed to observe changes and the interval following treatment for responses to occur vary depending on the desired effect. The particular amounts may be determined by conventional tests which are well known to the person skilled in the art.

The present invention relates in a ninth aspect to the antibody, the nucleic acid molecule, the set of two nucleic acid molecules, the vector, the set of two vectors or the host of the invention for use in a method of treating, inhibiting or diagnosing in vivo a tumor.

The tumor can be a benign or a malignant tumor and is preferably a malignant tumor. A malignant tumor is also referred to herein as cancer. The tumor is also preferably a solid tumor and the cancer preferably a solid cancer.

In accordance with a preferred embodiment of ninth aspect of the invention the tumor is prostate cancer.

As discussed herein above PSMA is a useful diagnostic and therapeutic target for tumors and in particular prostate cancer. Anti-PSMA antibodies being labeled with either diagnostic or therapeutic radionucleotides are already used in clinical practice for the diagnosis and radiotherapy of prostate cancer. Aberrant PSMA expression was not only found in prostate cancer but, for example, also in breast cancer (Kashoa et al. (2017), Clinical & Experimental Metastasis 34(4)).

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

### THE FIGURES SHOW

FIG. 1—Purification of antibody Fab fragments. Recombinant Fab proteins were purified by StrepTactin affinity chromatography and samples from individual fractions analyzed by reducing SDS-PAGE and Coomassie Brilliant Blue G-250 staining. Positions of the heavy and the light chains are marked by arrowheads and asterisks, respectively.

FIG. 2—SDS-PAGE analysis of heterologously expressed purified 5D3-hFab fragments. Elution fractions containing recombinant Fab proteins (purified by affinity chromatography) were pooled and concentrated to 1 mg/mL. 1 μg of each sample was separated by reducing SDS-PAGE and visualized by Coomassie Brilliant Blue G-250 staining. The final purity of individual Fab preparation is >95%.

FIG. 3—The thermal stability of Fab variants determined by nanoDSF. Protein samples were diluted in PBS/10% glycerol to the final concentration of 0.5 mg/mL and subjected to a temperature gradient of 20-95° C. (1.5° C./min). The first derivative of the intrinsic fluorescence change against the temperature is plotted. Curve maxima correspond to melting temperatures of individual proteins.

FIG. 4—Affinity of Fab variants determined by ELISA. Plates coated with recombinant PSMA were probed with two-fold dilution series of individual fragments and binding visualized using goat anti-human secondary antibody conjugated to horseradish peroxidase together with the TMB substrate. Binding curves were fitted using a non-linear regression algorithm.

FIG. 5—Affinity of Fab variants determined by flow cytometry. LNCaP (PSMA-positive) and DU145 (PSMA-negative) cell lines were probed with three-fold dilution series of tested Fab variants and bound antibodies visualized by the combination of anti-Strep and goat anti-mouse Alexa-Fluor647 antibodies. Cell samples were analyzed using a LSRFortessa flow cytometer and binding curves were fitted using a non-linear regression algorithm.

FIG. 6—Specificity of 5D3-hFabs determined by flow cytometry. LNCaP (PSMA-positive) and DU145 (PSMA-negative) cell lines were probed with three-fold dilution series of tested Fab variants and bound antibodies visualized by the combination of anti-Strep and goat anti-mouse Alexa-Fluor647 antibodies. Cell samples were analyzed using a LSRFortessa flow cytometer.

FIG. 7—Purification of full-length antibodies. Antibodies (IgG1 isotype) were purified by Protein A affinity chromatography and samples from individual fractions analyzed by reducing SDS-PAGE and Coomassie Brilliant Blue G-250 staining. Positions of the heavy and the light chains are marked by arrowheads and asterisks, respectively.

FIG. 8—SDS-PAGE analysis of heterologously expressed purified full-length antibodies. Elution fractions containing recombinant antibodies (purified by affinity chromatography) were pooled and concentrated to 1 mg/mL 1 μg of each sample was separated by non-reducing SDS-PAGE and visualized by Coomassie Brilliant Blue G-250 staining. The final purity of individual antibodies is >95%.

FIG. 9—The thermal stability of full-length antibodies determined by nanoDSF. Protein samples were diluted in PBS/3% glycerol to the final concentration of 0.5 mg/mL and subjected to a temperature gradient of 20-95° C. (1.5° C./min). The first derivative of the intrinsic fluorescence change against the temperature is plotted. Curve maxima correspond to melting temperatures of individual proteins.

FIG. 10—Affinity of full-length antibodies determined by ELISA. Plates coated with recombinant PSMA were probed with two-fold dilution series of individual antibodies and binding visualized using goat anti-human secondary antibody conjugated to horseradish peroxidase together with the TMB substrate. Binding curves were fitted using a non-linear regression algorithm.

FIG. 11—Specificity of full-length antibodies determined by flow cytometry. LNCaP (PSMA-positive) and DU145 (PSMA-negative) cell lines were probed with three-fold dilution series of tested full-length antibodies and bound antibodies visualized by goat anti-human Alexa-Fluor488 secondary antibody. Cell samples were analyzed using a LSRFortessa flow cytometer.

FIG. 12—In silico and structural analysis of humanized 5D3.

FIG. 13—SDS-PAGE analysis of optimized full-length antibodies. Elution fractions containing recombinant antibodies (purified by protein A affinity chromatography) were pooled and concentrated to 1 mg/mL. 1 μg of each sample was separated by non-reducing SDS-PAGE and visualized by Coomassie Brilliant Blue G-250 staining. The final purity of individual antibodies is >95%.

FIG. 14 —SDS-PAGE analysis of optimized full-length antibodies treated by PNGase F. Recombinant antibodies (purified by protein A affinity chromatography) were treated with PNGase F at 37° C. for 4 h and then separated by reducing SDS-PAGE and visualized by Coomassie Brilliant Blue G-250 staining. Differences in electrophoretic mobilities between the parent 5D3 and optimized variants prior to PNGase treatment reveal the presence or absence of the N-glycosylation site at the VH:N30 position. Thus, all versions except for the original antibody and the LVg2 mutant show higher mobility as they lack this N-glycosylation site. Upon PNGase deglycosylation, further increase in antibody mobility is due to the removal of oligosaccharides in the Fc portion.

FIG. 15—Thermal stability of optimized humanized 5D3 variants determined by nanoDSF. Protein samples were diluted in PBS/3% glycerol to the final concentration of 0.5 mg/mL and subjected to a temperature gradient of 20-95° C. (1.5° C./min). The first derivative of the intrinsic fluorescence change against the temperature is plotted. Curve maxima correspond to melting temperatures of individual proteins or domains.

FIG. 16—Affinities of optimized full-length antibodies determined by ELISA. Plates coated with recombinant PSMA were probed with two-fold dilution series of individual antibodies and binding was visualized using goat anti-human secondary antibody conjugated to horseradish peroxidase together with the TMB substrate. Binding curves were fitted using a non-linear regression algorithm.

FIG. 17—IFN-γ-activated U937 monocytes were mixed with streptavidin magnetic beads loaded with human PSMA in the presence of murine (m5D3) or humanized (h5D3) antibodies or without any antibody as a control (no Ab). Production of ROS was determined by quantifying chemiluminescence of added lucigenin. Note the markedly pronounced activation by h5D3 compared to m5D3.

The examples illustrate the invention.

### Examples

### 5D3-mFab Crystallization and the Design of Humanized 5D3 Models

The original Fab fragment of murine 5D3 (5D3-mFab) was cloned and heterologously expressed in insect S2 Schneider's cells as described previously (Novakova, Belousova et al. (2020), Int. J. Mol. Sci., 21(18):6672). The amino acid sequences of SEQ ID NOs 17 and 18 are the V_{H} region and the V_{L} region of the 5D3-mFab and the corresponding nucleotide sequences are SEQ ID NOs 19 and 20. The 5D3-mFab was crystallized using a hanging drop vapor diffusion setup with 100 mM Tris-HCl, 10 mM ZnSO₄, and 17.5% PEG 6000, pH 8, as a mother liquor solution. Diffraction data from a single crystal were collected upon vitrification from a mother liquor supplemented with 20% glycerol and the structure was solved by molecular replacement. The 5D3-mFab model was prepared using iterative cycles of the model building in COOT and restrained refinement in REFMAC5.

The refined 5D3-mFab structure served as template to generate humanized 5D3 variants using structure-based alignment and CDR grafting. Briefly, three structures with human framework sequences revealing the highest structural similarity to the 5D3-mFab were selected using the PDB eFold server (httpJ/www.ebi.ac.uk/msd-srv/ssm/). Sequences of V_{L} and V_{H} variable domains of 5D3-mFab were aligned with corresponding sequences of the search hits and positions and sequences of individual CDRs were determined according the Kabat nomenclature using the ANTICALIgN software (Jarasch, Kopp et al. (2016) Protein Eng. Des. Sel., 29(7)263-270; Jarasch, A. & Skerra, A. (2017) Aligning, analyzing, and visualizing sequences for antibody engineering: Automated recognition of immunoglobulin variable region features. Proteins 85, 65-71.). Models of humanized 5D3 were prepared by grafting the 5D3 CDRs onto framework regions of search hits. Additionally, some backmutations were introduced into each model based on a visual inspection of the aligned structures of the 5D3 variable domains and search hits in order to avoid local structural incompatibilities between CDRs of the mouse donor antibody and the human acceptor framework regions. In this manner, three humanized 5D3 models, denoted 2-5D3, 5-5D3, and 6-5D3, were prepared and further characterized.

### Cloning of Humanized 5D3-Fabs (5D3-hFab)

Optimized nucleotide sequences encoding the humanized 5D3-hFabs were designed by the backtranslation of their amino acid sequences resulting from the structural modelling as explained above and synthesized commercially (ThermoFisher Scientific). Genes encoding variable domains of 5D3-hFabs, flanked by the TwinStrep tag at the C-terminus of the CH1 constant domain, were cloned into a bicistronic expression vector using the Gibson assembly protocol. To this end, individual genes and backbone fragments were PCR amplified and assembled by Gibson Assembly Cloning Kit according to manufacturer's instructions (New England Biolabs). Nucleotide sequences of all expression constructs were verified by Sanger sequencing.

### Heterologous Expression of 5D3-hFabs

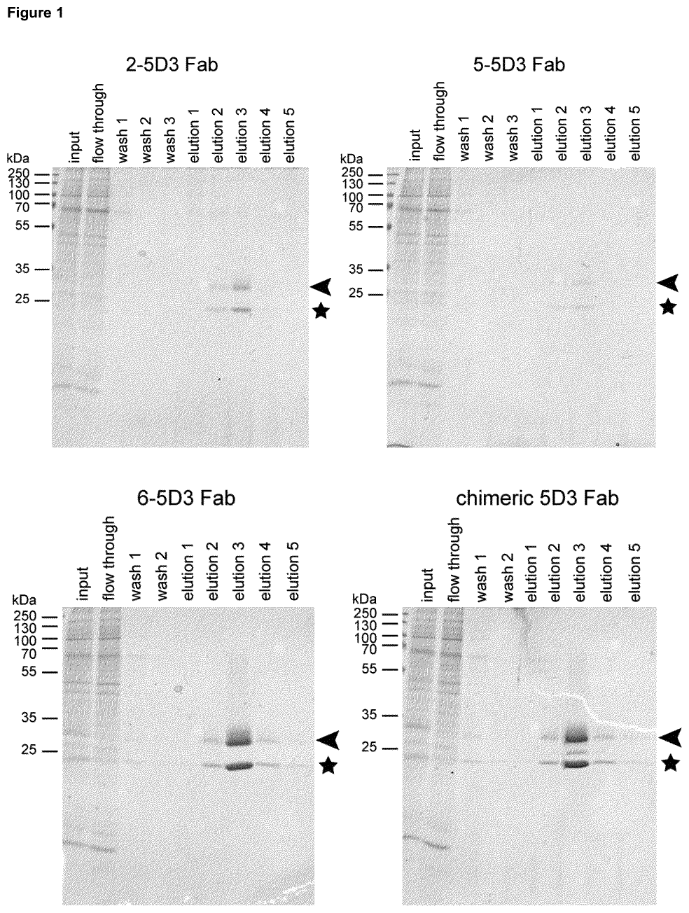

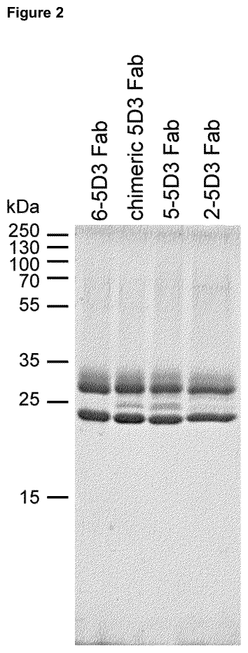

The resulting expression plasmids were introduced into HEK293T17 cells by transient transfection using a linear polyethyleneimine (Skultetyova, Ustinova et al. (2017), Sci Rep., 7(1):11547). Five days post transfection, the culture medium was harvested and cleared by centrifugation at 40 000×g for 20 min. The medium was filtered and constructs were purified via affinity chromatography on a StrepTactin XT column equilibrated with a purification buffer (50 mM Tris-HCl, 150 mM NaCl, 10 mM KCl, 10% glycerol, pH 8.0). The elution was carried out using the purification buffer supplemented with 5 mM D-biotin. Purification procedure is illustrated in FIG. 1. The highest expression levels were observed for 6-5D3-hFab, which were more than 10-fold higher compared to 2-5D3-hFab and 5-5D3-hFab, and comparable to the chimeric Fab variant (Table 1). Clearly, the 6-5D3-hFab variant retained advantageously high expression levels observed for parent murine and chimeric Fabs. Elution fractions were concentrated, filtered, and flash frozen in liquid nitrogen at concentrations approximately 2-4 mg/mL Aliquots were stored at −80° C. The final purity >90% was similar among all preparations (FIG. 2).

### Biophysical and Functional Characterization of 5D3-hFabs

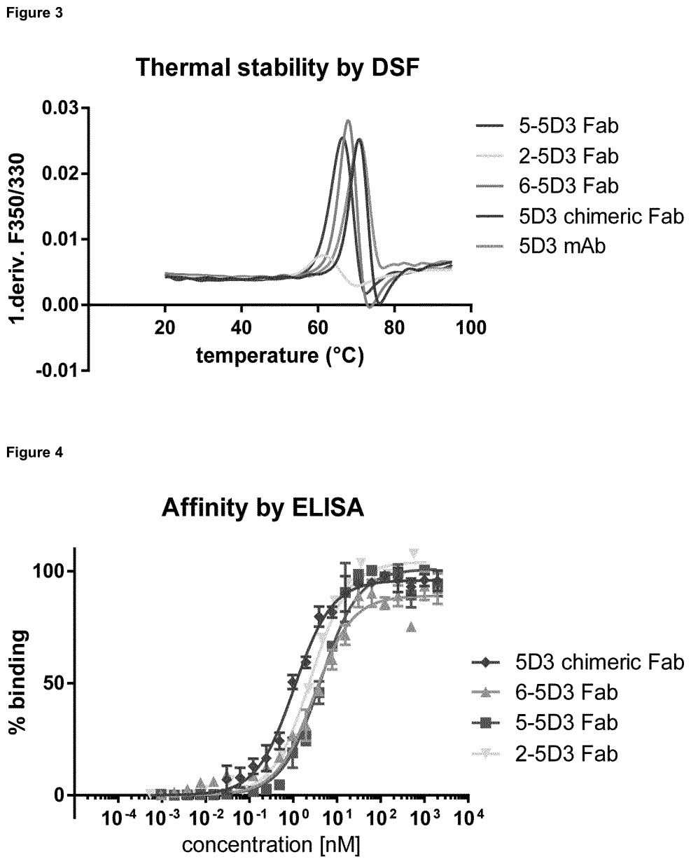

The temperature stability of 5D3-hFabs was determined by differential scanning fluorimetry (nanoDSF) using a Prometheus NT.48 fluorimeter (NanoTemper Technologies, Munchen, Germany). Protein samples were diluted in PBS/10% glycerol to final concentration of 0.5 mg/mL, loaded into measuring capillaries and subjected to a temperature gradient of 20-95° C., with the ramping temperature rate of 1.5° C./min. Intrinsic protein fluorescence curves at 330 nm and 350 nm were used to calculate melting temperatures of 5D3-hFabs and data are shown in FIG. 3 and corresponding Tm values in Table 1. The lowest stability of Tm=61.6° C. was observed for 2-5D3-hFab, which is 8.8° C. and 5.9° C. lower compared to 5-5D3-hFab and 6-5D3-hFab, respectively. These data suggest that 2-5D3-hFab might be ill suited for further development due to its limited stability.

Native ELISA was used to measure affinity of 5D3-hFabs for human PSMA. Experiments were performed in 96-well MaxiSorp plates coated with streptavidin complexed with biotin-tagged PSMA. Purified 5D3-hFabs were applied in 2-fold dilution series in the concentration range of 2000 nM-0.95 pM and goat anti-human secondary antibody conjugated to horseradish peroxidase, together with the TMB substrate, was used for 5D3-hFabs detection. Data were processed using a Prism 6 software (GraphPad, San Diego, CA) and binding curves fitted using an embedded non-linear curve-fitting algorithm (FIG. 4, Table 1). Affinity of all variants tested was in a single-digit nanomolar range (1.8-2.7 nM) and virtually identical to the parent murine and chimeric Fabs, documenting successful humanization results.

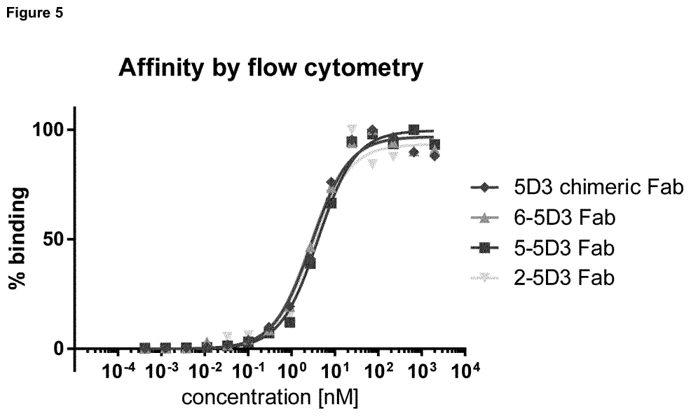

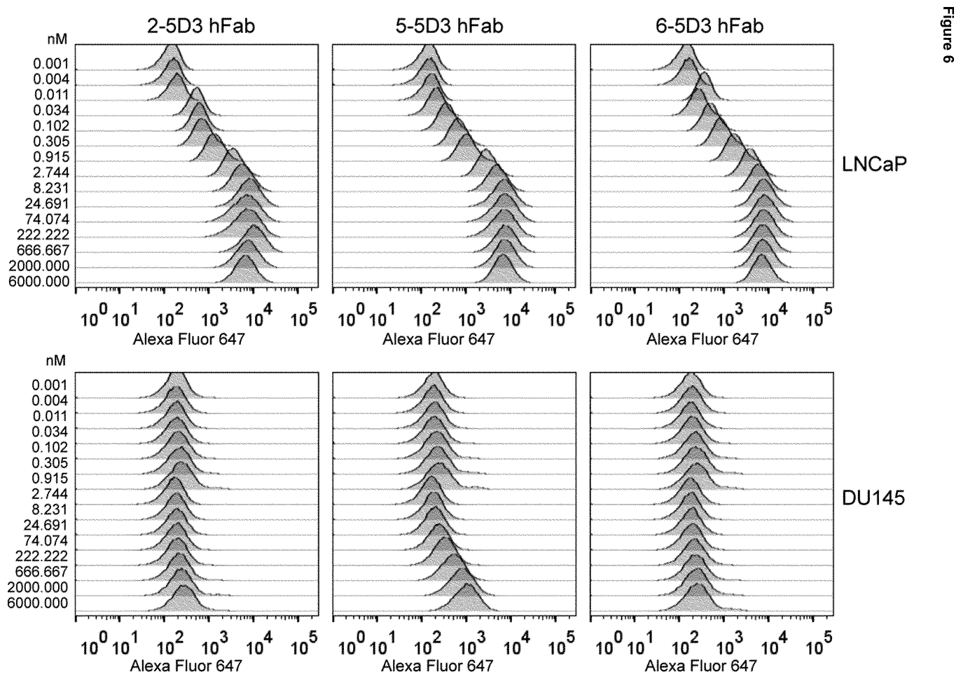

Flow cytometry was used to analyze affinity and specificity of 5D3-hFabs for PSMA in its physiological environment, i.e., at the surface of living cells. To this end, LNCaP and DU145 cells were used as representatives of PSMA-positive and PSMA-negative cell lines, respectively, in a protocol described previously (Novakova, Belousova et al. (2020), Int. J. Mol. Sci., 21(18):6672). Briefly, cells were treated by three-fold dilution series of 5D3-hFabs in the concentration range 6000 nM-0.42 pM. Bound 5D3-hFabs were detected by the combination of anti-Strep tag antibody and goat anti-mouse secondary antibody conjugated to Alexa Fluor 647. Cell samples were immediately analyzed using a LSRFortessa flow cytometer. All three 5D3-hFabs revealed similar affinity for cell-surface displayed PSMA in the range of 2.8-4.1 nM, confirming data from ELISA experiments (FIG. 5, Table 1). However, non-specific binding for DU145 cells was observed in the case of 5-5D3-hFab (FIG. 6) and such characteristics disqualified this clone from further development. On the other hand, the 2-5D3-hFab showed significantly diminished thermal stability.

TABLE 1

| | Yield | Stability | KD (nM; | KD (nM; | Specificity |
|---|---|---|---|---|---|
| Variant | (mg/L) | (° C.) | ELISA) | FC) | (FC) |
| 2-5D3-hFab | 0.6 | 61.6 | 2.7 | 3.0 | Y |
| 5-5D3-hFab | 0.2 | 70.4 | 1.8 | 4.1 | N |
| 6-5D3-hFab | 6.4 | 67.5 | 1.8 | 2.8 | Y |
| Chimeric Fab | 10.8 | 66.4 | 1.8 | 2.8 | Y |

The amino acid sequences of SEQ ID NOs 21/22, 25/26 and 1/2 are the V_{H} region and the V_{L} region of the exemplified versions 2, 5 and 6, respectively of 5D3-Fab and the corresponding nucleotide sequences are SEQ ID NOs 23/24, 27/28 and 9/10, respectively.

### Cloning, Expression and Purification of Full-Length Humanized Antibodies (2-5D3-IgG1, 5-5D3-IgG1, 6-5D3-IgG1)

To corroborate and extend findings on 5D3-hFabs, full-length humanized 5D3 variants were cloned, purified, and characterized in detail. To construct humanized 5D3-IgG1s, humanized variable domains were cloned into a bicistronic expression vector in frame with IgG1K human constant domains using the Gibson assembly protocol. The full-length humanized antibodies were denoted 2-5D3-IgG1, 5-5D3-IgG1, and 6-5D3-IgG1.

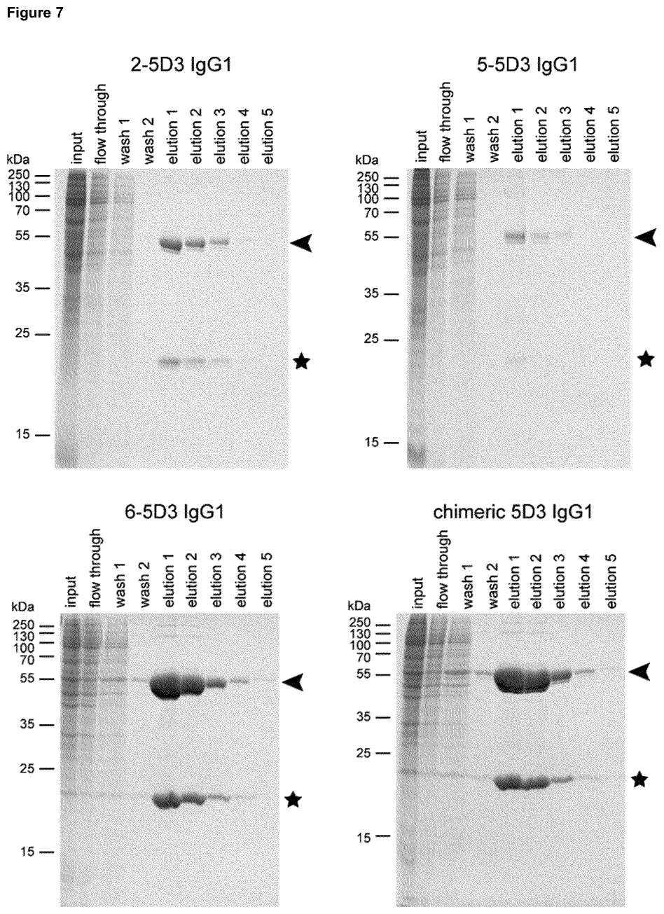

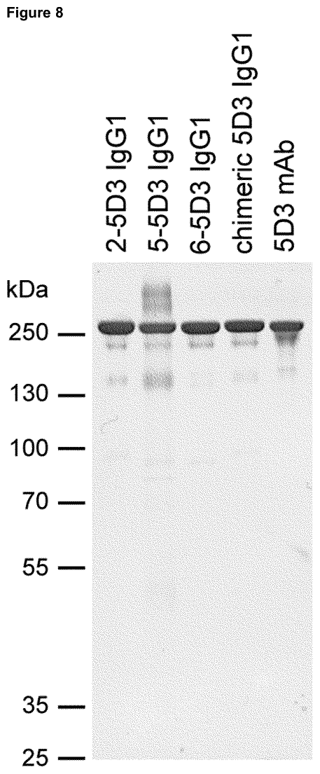

The resulting expression plasmids were introduced into HEK293T17 cells by transient transfection using a linear polyethyleneimine (Skultetyova, Ustinova et al. (2017), Sci. Rep. 7(1):11547). Five days post transfection, the culture medium was harvested and cleared by centrifugation at 40 000×g for 20 min. The medium was filtered, and constructs were purified via affinity chromatography on a protein A column equilibrated with phosphate-buffered saline, pH 7.4 (equilibration buffer). The elution was carried out using the elution buffer (100 mM glycine, pH 2.7), and elution fractions immediately neutralized with 1/10 volume of 1M Tris-HCl, pH 8.0. Purification procedure is illustrated in FIG. 7. The highest expression levels of 4.0 mg/L were observed for 6-5D3-IgG1, which were 5- to 10-fold higher compared to 2-5D3-IgG1 and 5-5D3-IgG1 (Table 2). Clearly, the 6-5D3-IgG1 variant retained advantageously high expression levels observed for parent murine and chimeric Fabs and chimeric IgG1. Elution fractions were concentrated, filtered, and flash frozen in liquid nitrogen at concentrations approximately 2-4 mg/mL with final purity >95% (FIG. 8).

### Biophysical and Functional Characterization of Humanized 5D3-IgG1s

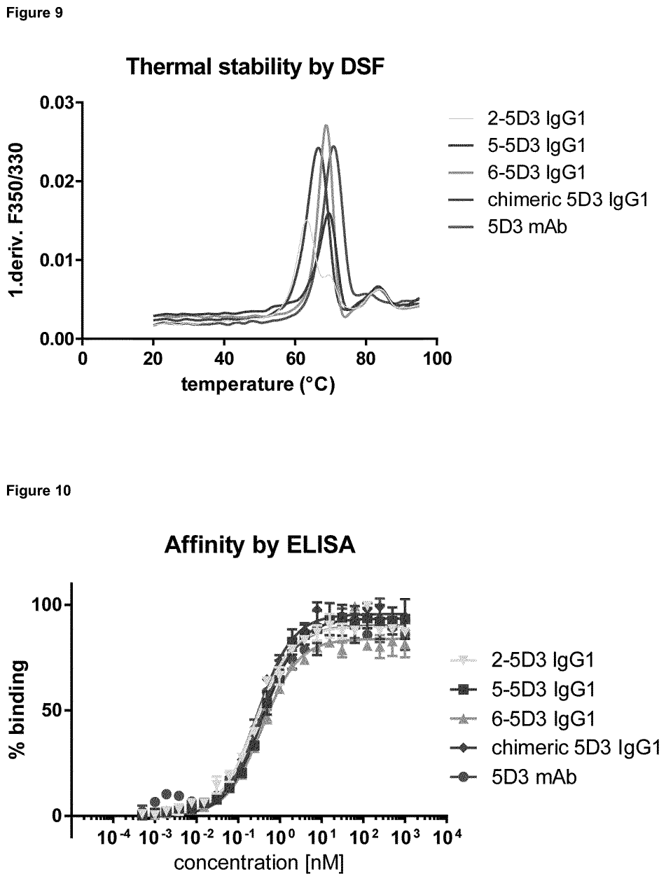

The temperature stability of humanized 5D3-IgG1s was determined by differential scanning fluorimetry (nanoDSF) as described above and derivatized melting curves together with corresponding Tm values shown in FIG. 9 and Table 2, respectively. The lowest stability of Tm=63.3° C. was observed for 2-5D3-IgG1, which is 5.9° C. and 5.3° C. lower compared to 5-5D3-IgG1 and 6-5D3-IgG1, respectively. In line with the inferior stability data for the 2-5D3-hFab shown above, these data suggest that 2-5D3-IgG1 might be ill suited for further development due to its limited stability.

Native ELISA was used to measure affinity of humanized 5D3-IgG1s for human PSMA in an experimental setup described above and binding curves together with corresponding KD values shown in FIG. 10 and Table 2, respectively. Affinity of all variants tested was in a picomolar range (260-380 pM) and virtually identical to the parent murine and chimeric full-length IgG1s, documenting successful outcome of the humanization process.

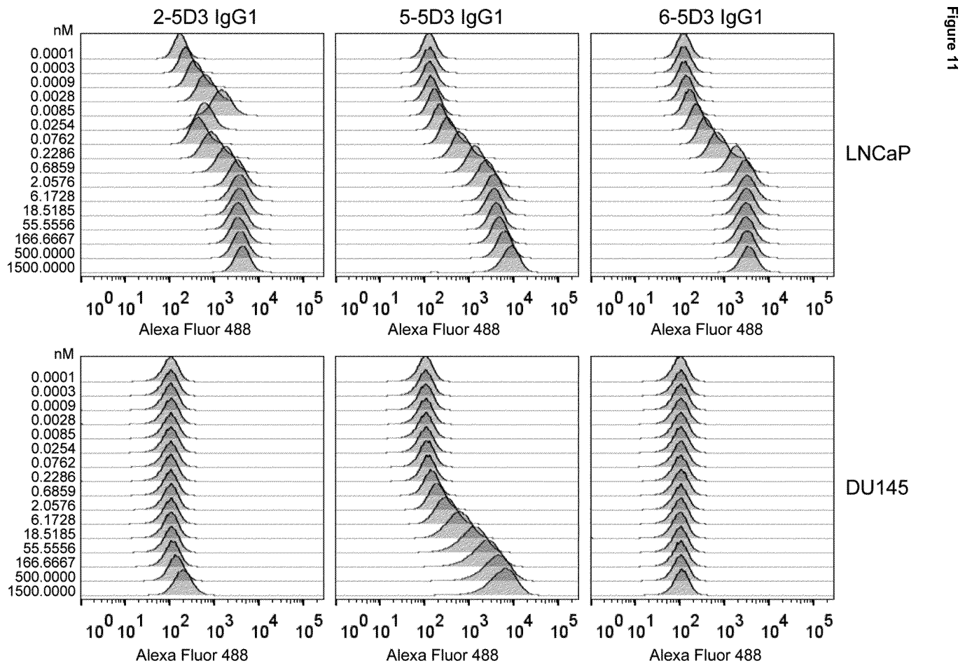

Flow cytometry was used to analyze affinity and specificity of humanized 5D3-IgG1s for PSMA in its physiological environment as described above and data are shown in FIG. 11 and Table 2. All three humanized 5D3-IgG1s revealed similar affinity for cell-surface displayed PSMA in the picomolar range (550-770 pM), confirming data from ELISA experiments. Importantly though, strong non-specific binding was observed in the case of DU145 cells for 5-5D3-IgG1 and precluding thus this clone from the further development.

TABLE 2

| | Yield | Stability | KD (nM; | KD (nM; | Specificity |
|---|---|---|---|---|---|
| Variant | (mg/L) | (° C.) | ELISA) | FC) | (FC) |
| 2-5D3-IgG1 | 0.7 | 63.3 | 0.26 | 0.64 | Y |
| 5-5D3-IgG1 | 0.3 | 69.2 | 0.39 | 0.77 | N |
| 6-5D3-IgG1 | 4.0 | 68.6 | 0.38 | 0.56 | Y |
| Chimeric 5D3 | 4.1 | 66.5 | 0.28 | 0.35 | Y |
| IgG1 | | | | | |

Overall, as documented by the above examples, out of three humanized variants of 5D3 murine antibody, the 6-5D3-IgG1 (6-5D3-hFab) performs outstandingly well as it is the only one that retained high expression yields, temperature stability and specificity of the parent 5D3 mAb. It was unexpected that a humanized variant of 5D3 murine antibody can be obtained which retains these three favourable characteristics of the parent 5D3 mAb despite the CDR-grafting during humanization.

Identification of Sequence Liabilities in h5D3

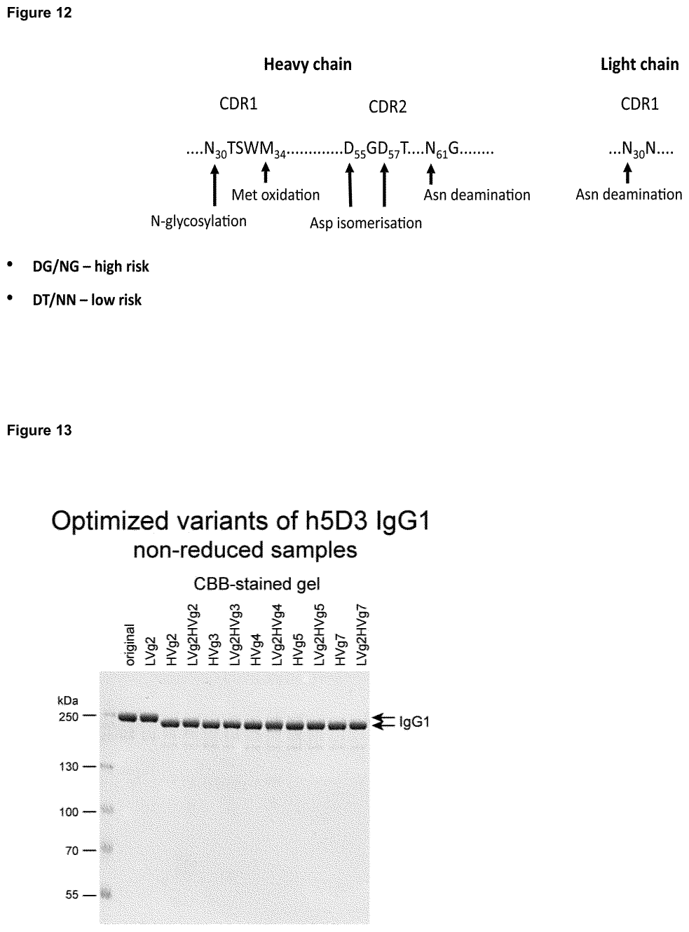

For the antibody therapeutics development, it is imperative that the lead mAb has a limited sequence liability load to increase changes of successful project completion and decrease the attrition rate. It is thus common to assess biophysical and biochemical properties of materials at the discovery stage to avoid or reduce development liabilities. We used in silico prediction to assess liabilities of humanized 5D3. It has been found that h5D3 has a high sequence liability load, including a N-glycosylation (N30-T31-S32) motif in CDR-H1, a high-risk aspartate isomerization site (D55-G56), and a high-risk asparagine deamidation site (N61-G62) in CDR-H2. For example, N-glycosylation in CDR is not desired for the development of a therapeutic antibody as it will likely have a strong impact on biological activity and cannot be adequately controlled during manufacture process. Furthermore, low risk sites were identified at positions VH:M34 (methionine oxidation), VH:D57-T58 (aspartate isomerization), and VL:N30-N31 (asparagine deamidation) (see FIG. 12, Table 3).

Using the AlphaFold prediction tool we also created an in-silico model of the h5D3 VH-V_{L} dimer to allow for visual inspection of the affected sites and their liabilities as well as to assist us in the rational design of suitable mutations. For the VH:M34 site, the methionine side chain is buried within the protein structure and thus unlikely to be prone to oxidation. Similarly, the VH:D57-T58 pair is a part of a β-sheet secondary structural motif. As positional flexibility is needed for “effective” aspartate isomerization it is unlikely that pronounced D57 isomerization will be observed. With the exception of the N61 site chain, the loops harboring VH:N30-T31-S32, VH:D55-G56, VH:N61-G62, and VL:N30-N31 sites are solvent exposed and thus amenable to site-directed mutagenesis. The structural analysis therefore already indicates that mutations designed to mitigate the identified sequence liabilities are tolerated.

h5D3 Variants Addressing the Identified Liabilities

Selected h5D3 mutants (Table 3) were thus designed to address liabilities identified. Importantly, the N-glycosylation site of the original murine 5D3 as well as humanized 5D3 would be associated with development liabilities, yet functional effects of the removal of this site were not apparent. The mutated variants were prepared by either de novo gene synthesis or QuikChange site directed mutagenesis, expressed in HEK293 cells, and purified by protein A affinity chromatography as described for the parent h5D3 antibody (FIG. 13). Purified proteins were next assayed for the thermal stability and binding affinity against human PSMA (ELISA) as described for the parent h5D3 antibody.

TABLE 3
Sequence liabilities and proposed mutations to remove the liabilities.
Liabilities

| Sequence liability | Type of liability |
|---|---|
| VH: N30-T31-S32 | N-glycosylation |
| VH: M34 | Methionine oxidation |
| VH: D55-G56 | Aspartate isomerization (high risk) |
| VH: D57-T58 | Aspartate isomerisation (low risk) |
| VH: N61-G62 | Asparagine deamination (high risk) |
| VL: N30-N31 | Asparagine deamination (low risk) |

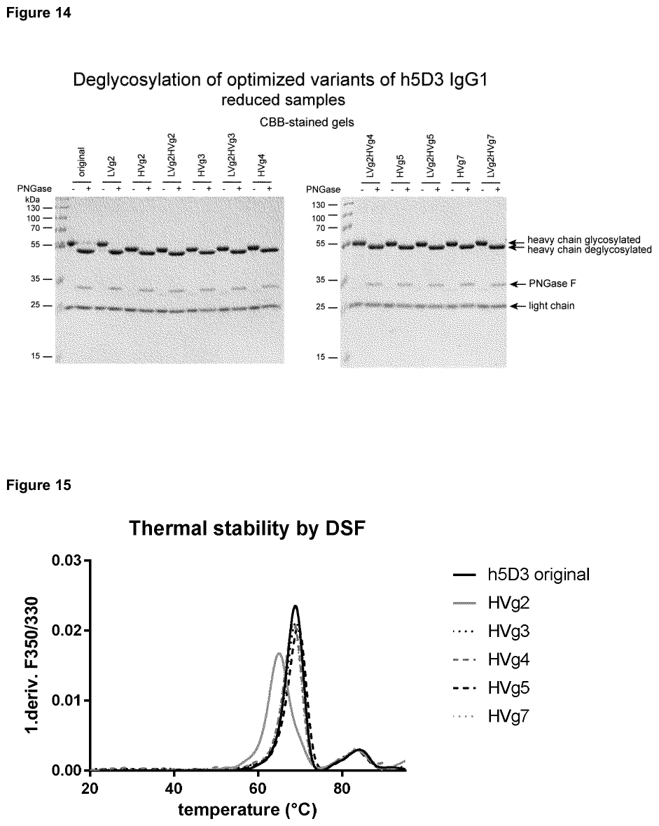

Optimized humanized 5D3 variants were expressed in suspension HEK293T cells and purified to near homogeneity (see FIG. 13). All optimized humanized 5D3 were obtained in satisfactory amounts. The most importantly, optimized variants of the heavy chain reveal higher mobility in SDS-PAGE due to the absence of N-glycosylation, as all optimized variants carry the HV:N30Q mutation. The presence of N-glycosylation at the HV:N30Q was also verified by the treatment of all variants by PNGase F and comparing their electrophoretic mobilities by SDS-PAGE (FIG. 14).

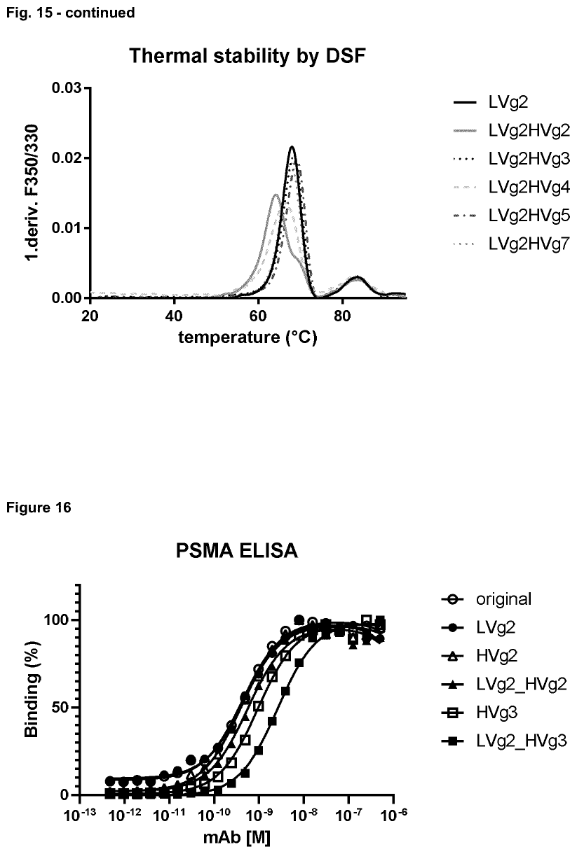

The thermal stability of the optimized humanized 5D3 variants was tested (FIG. 15, Table 4). All optimized humanized 5D3 essentially retained the thermal stability of humanized 5D3. The thermal stability as determined by nanoDSF (Nano differential scanning fluorimetry) was between 64.1° C. and 68.8° C.

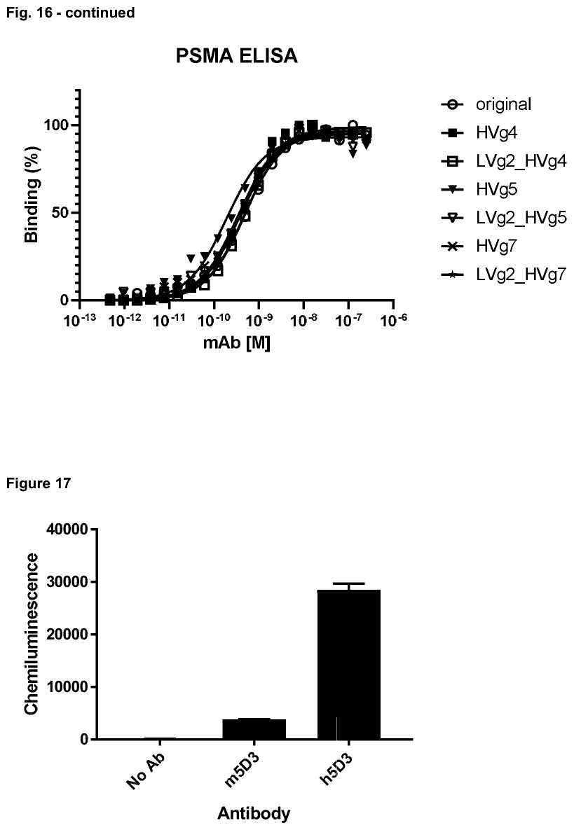

Also, the affinity of the optimized humanized 5D3 variants was determined by ELISA (FIG. 16, Table 4). The affinity as determined by ELISA was below 3 nM for all optimized humanized 5D3 variants.

TABLE 4
Summary of tested properties of variants of h5D3

| h5D3 variant | Mutations | Tm (° C.) | ELISA (nM) |
|---|---|---|---|
| original | none | 68.7 | 0.25 ± 0.046 |
| LVg2 | LV: N30E | 67.8 | 0.29 |
| HVg2 | HV: N30Q, G56A, | 65.0 | 0.34 |
| | G62A | | |
| HVg3 | HV: N30Q, D55E, | 68.8 | 0.93 |
| | D57E, G62A | | |
| HVg4 | HV: N30Q, D55E, | 68.3 | 0.36 |
| | G62A | | |
| HVg5 | HV: N30Q, D55E, | 69.3 | 0.06 |
| | D57S, G62A | | |
| HVg7 | HV: N30Q | 68.2 | 0.35 |
| LVg2HVg2 | HV: N30Q, G56A, | 64.1 | 0.47 |
| | G62A; LV: N30E | | |
| LVg2HVg3 | HV: N30Q, D55E, | 68.2 | 2.69 |
| | D57E, G62A; | | |
| | LV: N30E | | |
| LVg2HVg4 | HV: N30Q, D55E, | 66.3 | 0.50 |
| | G62A; LV: N30E | | |
| LVg2HVg5 | HV: N30Q, D55E, | 68.9 | 0.20 |
| | D57S, G62A; | | |
| | LV: N30E | | |
| LVg2HVg7 | HV: N30Q; | 67.6 | 0.37 |
| | LV: N30E | | |

The above data clearly show that mutations and combinations thereof are permissible at sites with identified liabilities resulting in 5D3 variants with biophysical characteristics matching the parent antibody but improved developability.

Quantification of the Reactive Oxygen Species (ROS) Production: Comparison of Murine 5D3 (n5D3) and Humanized 5D3 (h5D3)

Immunotherapy provides a revolutionizing breakthrough in cancer treatment using monoclonal antibodies targeting cancer cells expressing tumor-associated antigens. Such antibodies bind and activate immune cells resulting in tumor cell apoptosis and elimination via antibody-dependent cellular cytotoxicity (ADCC) mechanisms. ADCC is a mechanism whereby the immune effector cells (such as macrophages, NK cells, or T-cells) actively lyse target tumor cells, whose membrane-surface antigens have been bound by specific antibodies.

Macrophages are one of the most abundant immune cells found in tumor microenvironment and upon stimulation can exert cytotoxic functions against target cancer cells via the release of reactive oxygen species (ROS), nitrogen radicals, pro-inflammatory cytokines, such as TNF-α and IL-1β, antigen presentation and phagocytosis (https//doi.org/10.1002/kjm2.12405). Macrophages and monocytes express FCγRI (CD64) receptors on their plasma membrane and CD64-mediated effector functions are essential for therapeutic efficacy of mAb therapy in patients with cancer. Expression levels of CD64 are stimulated by the treatment of the cells by IFN-γ (dx.doi.org/10.1021rja509513c). For ADCC, a therapeutic antibody engages simultaneously both CD64 and a target receptor at the surface of cancer cells resulting in their cross-linking followed by the activation of the tyrosine kinases leading to both production of ROS by effector cells as well as internalization of the opsonized particles larger than 0.5 μm (e.g., target cancer cells). (J Immunol Jun. 15, 1999, 162 (12) 7041-7048) (https-J/doi.org/10.3389/fimmu.2021.734229).

Quantification of the Reactive Oxygen Species (ROS) production by the macrophages was carried out using the U937 monocytic cell line cultured in the RPMI 1640 media+10% FBS without Phenol Red. Cells were treated with 10 ng/ml IFN-γ for three days prior to experiment, to increase the number of FCγRI at the plasma membrane. Streptavidin magnetic beads (1 μm, Roche) were loaded with biotin-labelled PSMA (1 mg/ml) for 30 minutes and remaining biotin binding sites were blocked with D-biotin for 15 minutes. 5D3 antibodies were then attached to the PSMA coated beads. IFN-γ-treated U937 cells (3×10⁶ cells/ml) were mixed with beads coated with antibodies (with beads/naked antibodies as a control) along with 2.5 mM Lucigenin (Sigma) in a final volume of 100 μL in a 96-well white U-shaped plate. The plate was immediately centrifuged for 2 minutes at 200 xg, and lucigenin chemiluminescence (as a measure of the ROS production) quantified using a Clariostar luminometer. The data were plotted using Graph Pad prism software.

IFN-γ-activated U937 monocytes were mixed with streptavidin magnetic beads loaded with human PSMA in the presence of murine (m5D3) or humanized (h5D3) antibodies or without any antibody as a control (no Ab). Production of ROS was determined by quantifying chemiluminescence of added lucigenin. h5D3 advantageously displays a markedly pronounced activation as compared to m5D3 (FIG. 17).

1. An antibody that binds prostate-specific membrane antigen (PSMA), wherein the antibody comprises
the V_{H} region determined by the amino acid sequence of
(SEQ ID NO: 30)
EVQLVESGGGLVQPGGSLRLSCAASGYAF(X₁)(X₂)(X₃)W(X₄)NWVR
QAPGKGLEWISRIYPG(X₅)(X₆)(X₇)(X₈)NY(X₉)(X₁₀)KFKGKATI
SADKSKNTLYLQMNSLRAEDTAVYYCARGEWYLYYFDYWGQGTLVTVS
S,
wherein
for (X₁) to (X₃) at least one applies:
(X₁) is G, A, V, L, I, Y, H, K, R, Q, N, E, D, S or T,
(X₂) is P or T, and
(X₃) is G, A, V, L, I, Y, H, K, R, Q, N, E, D, T or S,
with the proviso that (X₁) to (X₃) are not N-(Z)-S/T, wherein (Z) can be any amino acid but not P, or
wherein (X₁) to (X₃) are N-T-S;
(X₄) is M, I or L;
for (X₅) and (X₆) at least one applies:
(X₇) is G, A, V, L, I, Y, H, Q, N, E, D, S or T, and
(X₈) is G, A, V, L, I, H, K, R, Q, N, E, D, S or T,
with the proviso that (X₅)-(X₆) are not D-S, N-G or N-S;
for (X₇) and (X₈) at least one applies:
(X₇) is G, A, V, L, I, Y, H, Q, N, E, D, S or T, and
(X₈) is G, A, V, L, H, Q, N, E, D, S or T,
with the proviso that (X₇)-(X₈) are not D-G, D-S, N-G or N-S;
for (X₉) and (X₁₀) at least one applies:
(X₉) is G, A, V, L, I, Y, H, K, R, Q, N, E, D, S or T, and
(X₁₀) is G, A, V, L, I, Y, H, K, R, Q, N, E, D, S or T,
with the proviso that (X₉)-(X₁₀) are not D-G, D-S or N-S;
or an amino acid sequence being at least 90% identical thereto; and
the V_{L}region determined by the amino acid sequence of
(SEQ ID NO: 31)
DIQMTQSPSSLSASVGDRVTITCSASQGI(X₁₁)(X₁₂)FLTWYQQKPGK
ALKLLIYYTSSLHSGVPSRFSGSGSGTDYTLTISSLQPEDFATYYCQQY
SNLPFTFGQGTKVEIK,
wherein
for (X₁₁) and (X₁₂) at least one applies:
(X₁₁) is G, A, V, L, I, Y, H, K, R, Q, N, E, D, S or T, and
(X₁₂) is G, A, V, L, I, Y, H, K, R, Q, N, E, D, S or T,
with the proviso that (X_{II})-(X₁₂) are not D-G, D-S, N-G or N-S;
or an amino acid sequence being at least 90% identical thereto,
provided that
the three CDRs of the V_{H} region are determined by the amino acid sequences of GYAF(X₁)(X₂)(X₃)W(X₄)N (SEQ ID NO: 32), wherein
for (X₁) to (X₃) at least one applies:
(X₁) is G, A, V, L, I, Y, H, K, R, Q, N, E, D, S or T,
(X₂) is P or T, and
(X₃) is G, A, V, L, I, Y, H, K, R, Q, N, E, D, T or S,
with the proviso that (X₁) to (X₃) are not N-(Z)-S/T, wherein (Z) can be any amino acid but not P, or
wherein (X₁) to (X₃) are N-T-S; and
(X₄) is M, I or L,
RIYPG(X₅)(X₆)(X₇)(X₈)NY(X₉)(X₁₀)KFK (SEQ ID NO: 33), wherein
for (X₅) and (X₆) at least one applies:
(X₅) is G, A, V, L, I, Y, H, Q, N, E, D, S or T, and
(X₆) is G, A, V, L, I, H, K, R, Q, N, E, D, S or T,
with the proviso that (X₅)-(X₆) are not D-S, N-G or N-S;
for (X₇) and (X₈) at least one applies:
(X₇) is G, A, V, L, I, Y, H, Q, N, E, D, S or T, and
(X₈) is G, A, V, L, H, Q, N, E, D, S or T,
with the proviso that (X₇)-(X₈) are not D-G, D-S, N-G or N-S;
for (X₉) and (X₁₀) at least one applies:
(X₉) is G, A, V, L, I, Y, H, K, R, Q, N, E, D, S or T, and
(X₁₀) is G, A, V, L, I, Y, H, K, R, Q, N, E, D, S or T,
with the proviso that (X₉)-(X₁₀) are not D-G, D-S or N-S; and
SEQ ID NO: 5, and
the three CDRs of the V_{L} region are determined by the amino acid sequences of SASQGI(X₁₁)(X₁₂)FLT (SEQ ID NO: 34), wherein
for (X₁₁) and (X₁₂) at least one applies:
(X₁₁) is G, A, V, L, I, Y, H, K, R, Q, N, E, D, S or T, and
(X₁₂) is G, A, V, L, I, Y, H, K, R, Q, N, E, D, S or T,
with the proviso that (X₁₁)-(X₁₂) are not D-G, D-S, N-G or N-S;
SEQ ID NO: 7, and
SEQ ID NO: 8.

2. The antibody of claim 1, wherein the antibody comprises
a V_{H}region differing by no more than 5 amino acid substitutions from SEQ ID NO: 30, and/or
a V_{L}region differing by no more than 5 amino acid substitutions from SEQ ID NO: 31.

3. The antibody of claim 2, wherein the amino acid substitutions are conservative amino acid substitutions.

4. The antibody according to claim 1, wherein the PSMA is determined by the amino acid sequence of SEQ ID NO: 29.

5. The antibody according to claim 1, lacking an N-glycosylation site in the V_{H} region, with the proviso that (X₁) to (X₃) are not N-(Z)-S/T, wherein (Z) can be any amino acid but not P.

6. The antibody according to claim 1, wherein said antibody is coupled to
(a) a labelling group,
(b) a toxin,
(c) a drug,
(d) a radionucleotide,
(e) a photosensitizing functional group
(f) a cytokine,
(g) a chemokine,
(h) an enzyme,
(i) a component modulating serum half-life
(j) an antibody or an Fc part thereof, or
(k) an antibody mimetic.

7. The antibody according to claim 6, wherein the antibody mimetic is an anticalin.

8. A nucleic acid molecule encoding the antibody according to claim 1.

9. A set of two nucleic acid molecules comprising a first nucleic acid molecule and a second nucleic acid molecule, wherein the first nucleic acid molecule encodes the V_{H} region of the antibody according to claim 1 and the second nucleic acid molecule encodes the V_{L} region of the antibody according to claim 1.

10. A vector comprising the nucleic acid molecule of claim 8 or the set of two nucleic acid molecules of claim 9 in an expressible form.

11. A set of two vectors comprising a first vector and a second vector, wherein the first vector comprises the first nucleic acid molecule of claim 9 in an expressible form and the second vector comprises the second nucleic acid molecule of claim 9 in an expressible form, and wherein the first and/or second vector optionally comprises a proteinaceous compound as defined by any one of a labelling group, a toxin, a drug, a cytokine, a chemokine, an enzyme, a component modulating serum half-life, an antibody or an Fc part thereof, or an antibody mimetic in an expressible form.

12. A non-human host comprising nucleic acid molecule of claim 8.

13. A method for producing an antibody of claim 1, comprising
(a) culturing a non-human host comprising a nucleic acid molecule encoding said antibody under conditions that allow synthesis of said antibody; and
(b) recovering the produced antibody.

14. A diagnostic composition comprising any one of the antibody of claim 1, the nucleic acid molecule of claim 8, the set of two nucleic acid molecules of claim 9, the vector of claim 10, the set of two vectors of claim 11, or the host of claim 12.

15-16. (canceled)

17. A vector comprising the nucleic acid molecule of claim 8 or the set of two nucleic acid molecules of claim 9 in an expressible form and a proteinaceous compound as defined by any one of a labelling group, a toxin, a drug, a cytokine, a chemokine, an enzyme, a component modulating serum half-life, an antibody or an Fc part thereof, or an antibody mimetic in an expressible form.

18. A non-human host comprising the set of two nucleic acid molecules of claim 9.

19. A non-human host comprising the vector of claim 10.

20. A non-human host comprising the set of two vectors of claim 11.

21. A pharmaceutical composition comprising any one of the antibody of claim 1, the nucleic acid molecule of claim 8, the set of two nucleic acid molecules of claim 9, the vector of claim 10, the set of two vectors of claim 11, or the host of claim 12.

22. A method of treating, inhibiting, or diagnosing in vivo a tumor comprising using the antibody of claim 1, the nucleic acid molecule of claim 8, the set of nucleic acid molecules of claim 9, the vector of claim 10, the set of two vectors of claim 11, or the non-human host of claim 12.

23. The method of claim 22, wherein the tumor is prostate cancer.


SEQUENCE LISTING (34 sequences)

SEQ ID NO: 1 - protein, 119 aa, Artificial Sequence
  Humanized 5D3-6 - heavy chain
EVQLVESGGGLVQPGGSLRLSCAASGYAFNTSWMNWVRQAPGKGLEWISRIYPGDGDTNYNGKFKGKATISADKSKNTLYLQMNSLRAEDTAVYYCARGEWYLYYFDYWGQGTLVTVSS

SEQ ID NO: 2 - protein, 107 aa, Artificial Sequence
  Humanized 5D3-6 - light chain
DIQMTQSPSSLSASVGDRVTITCSASQGINNFLTWYQQKPGKALKLLIYYTSSLHSGVPSRFSGSGSGTDYTLTISSLQPEDFATYYCQQYSNLPFTFGQGTKVEIK

SEQ ID NO: 3 - protein, 10 aa, Artificial Sequence
  Humanized 5D3-6 - heavy chain
GYAFNTSWMN

SEQ ID NO: 4 - protein, 17 aa, Artificial Sequence
  Humanized 5D3-6 - heavy chain
RIYPGDGDTNYNGKFKG

SEQ ID NO: 5 - protein, 10 aa, Artificial Sequence
  Humanized 5D3-6 - heavy chain
GEWYLYYFDY

SEQ ID NO: 6 - protein, 11 aa, Artificial Sequence
  Humanized 5D3-6 - light chain
SASQGINNFLT

SEQ ID NO: 7 - protein, 7 aa, Artificial Sequence
  Humanized 5D3-6 - light chain
YTSSLHS

SEQ ID NO: 8 - protein, 9 aa, Artificial Sequence
  Humanized 5D3-6 - light chain
QQYSNLPFT

SEQ ID NO: 9 - DNA, 357 bp, Artificial Sequence
  Humanized 5D3-6 - heavy chain
gaagttcaactggtggaaagcggtggtggtctggttcagcctggtggtagcctgcgtctgagctgtgcagcaagcggttatgcatttaataccagctggatgaattgggttcgtcaggcacctggtaaaggtctggaatggattagccgtatttatcctggtgatggtgataccaactataacggtaaattcaaaggcaaagcaaccatcagcgcagacaaaagcaaaaataccctgtacctgcaaatgaatagtctgcgtgcagaggataccgcagtgtattattgtgcacgtggtgaatggtatctgtactattttgattattggggtcagggcaccctggtcaccgtctcctca

SEQ ID NO: 10 - DNA, 321 bp, Artificial Sequence
  Humanized 5D3-6 - light chain
gacatccagatgacccagagtccgagcagcctgagcgcaagcgttggtgatcgtgttaccattacctgtagcgccagccagggtattaacaattttctgacctggtatcagcagaaaccgggtaaagcactgaaactgctgatctattataccagcagcctgcatagcggtgttccgagccgttttagcggtagcggtagtggcaccgattataccctgaccattagcagtctgcaaccggaagattttgcaacctattattgtcagcagtatagcaatctgccgtttacctttggtcagggcaccaaagtcgagatcaaa

SEQ ID NO: 11 - DNA, 30 bp, Artificial Sequence
  Humanized 5D3-6 - heavy chain
ggttatgcatttaataccagctggatgaat

SEQ ID NO: 12 - DNA, 51 bp, Artificial Sequence
  Humanized 5D3-6 - heavy chain
cgtatttatcctggtgatggtgataccaactataacggtaaattcaaaggc

SEQ ID NO: 13 - DNA, 30 bp, Artificial Sequence
  Humanized 5D3-6 - heavy chain
ggtgaatggtatctgtactattttgattat

SEQ ID NO: 14 - DNA, 33 bp, Artificial Sequence
  Humanized 5D3-6 - light chain
agcgccagccagggtattaacaattttctgacc

SEQ ID NO: 15 - DNA, 21 bp, Artificial Sequence
  Humanized 5D3-6 - light chain
tataccagcagcctgcatagc

SEQ ID NO: 16 - DNA, 27 bp, Artificial Sequence
  Humanized 5D3-6 - light chain
cagcagtatagcaatctgccgtttacc

SEQ ID NO: 17 - protein, 119 aa, Artificial Sequence
  5D3 mAb - heavy chain
EVQLQQSGPELVKPGASVKISCKTSGYAFNTSWMNWVKQRPGQGLEWIGRIYPGDGDTNYNGKFKGKATLTADKSSSTAHMHLSSLTSVDSAVYFCARGEWYLYYFDYWGQGTTLTVSS

SEQ ID NO: 18 - protein, 107 aa, Artificial Sequence
  5D3 mAb - light chain
DIQMTQTTSSLSASLGDRVTISCSASQGINNFLTWYQQKPDGTLKLLIYYTSSLHSGVPSRFSGSGSGTDYSLTIRNLEPEDIATYYCQQYSNLPFTFGGGTKVEIK

SEQ ID NO: 19 - DNA, 357 bp, Artificial Sequence
  5D3 mAb - heavy chain
gaggttcagcttcagcagtctggacctgaactggtgaagcctggggcctcagtgaagatttcctgcaaaacttctggctacgcattcaatacctcttggatgaactgggtgaagcagaggcctggacagggtcttgagtggattggacggatttatcctggagatggagatactaactacaatgggaagttcaagggcaaggccacactgactgcagacaaatcctccagcacagcccacatgcacctcagcagtctgacctctgtggattctgcggtctatttctgtgcaagaggagaatggtacctttactactttgactactggggccaaggcaccactctcacagtctcctca

SEQ ID NO: 20 - DNA, 321 bp, Artificial Sequence
  5D3 mAb - light chain
gacatccagatgacccagacaacatcctccctgtctgcctctctgggagacagagtcaccatcagttgcagtgcaagtcagggcattaacaattttttaacctggtatcagcagaaaccagatggaactcttaaactcctgatctattacacatcaagtttacactcaggagtcccatcaaggttcagtggcagtgggtctgggacagattattctctcaccattaggaacctggaacctgaagatattgccacttactattgtcagcaatatagtaaccttccgttcacgttcggaggggggaccaaggtggaaataaaa

SEQ ID NO: 21 - protein, 119 aa, Artificial Sequence
  Humanized 5D3-2 - heavy chain
QVQLVQSGAEVKKPGASVKVSCKASGYAFNTSWMNWVRQAPGQGLEYIGRIYPGDGDTNYNGKFKGKATMTADKSISTVYMEVRRLRSDDTAVYYCARGEWYLYYFDYWGQGTLVTVSS

SEQ ID NO: 22 - protein, 107 aa, Artificial Sequence
  Humanized 5D3-2 - light chain
DIQMTQSPSSLSASVGDRVTITCSASQGINNFLTWYQHEPGKALKLLIYYTSSLHSGVPSRFSGSGSGTDYTLTISTLQPEDFATYYCQQYSNLPFTFGGGTKVEIK

SEQ ID NO: 23 - DNA, 357 bp, Artificial Sequence
  Humanized 5D3-2 - heavy chain
caagttcaactggtgcagagcggtgccgaagttaaaaaaccgggtgcaagcgttaaagttagctgtaaagcaagcggttatgcctttaataccagctggatgaattgggttcgtcaggcacctggtcaaggtctggaatacattggtcgtatttatcctggtgatggcgataccaactataacggtaaattcaaaggtaaagcaaccatgaccgcagataaaagcattagcaccgtttatatggaagttcgtcgtctgcgtagtgatgataccgcagtgtattattgtgcacgtggtgaatggtatctgtactattttgattattggggtcagggcaccctggtcaccgtctcctca

SEQ ID NO: 24 - DNA, 321 bp, Artificial Sequence
  Humanized 5D3-2 - light chain
gacatccagatgacccagagtccgagcagcctgagcgcaagcgttggtgatcgtgttaccattacctgtagcgccagccagggtattaacaattttctgacctggtatcagcatgaaccgggtaaagcactgaaactgctgatctattataccagcagcctgcatagcggtgttccgagccgttttagcggtagcggtagtggcaccgattataccctgaccattagcaccctgcagccggaagattttgcaacctattattgtcagcagtatagcaatctgccgtttacctttggtggtggcaccaaagtcgagatcaaa

SEQ ID NO: 25 - protein, 119 aa, Artificial Sequence
  Humanized 5D3-5 - heavy chain
QVQLVQSGPEVKKPGASVRLSCKASGYAFNTSWMNWVRQAPGQGLEWIGRIYPGDGDTNYNGKFKGKATMTADKSTNTAYMELRSLRSDDTAVYYCGRGEWYLYYFDYWGQGTLVTVSS

SEQ ID NO: 26 - protein, 107 aa, Artificial Sequence
  Humanized 5D3-5 - light chain
DIQMTQSPSSVSASVGDRVTISCSASQGINNFLTWYQQKLGKALQLLIYYTSSLHSGVPSRFSGSGSGTDYTLTISSLQPEDFATYYCQQYSNLPFTFGPGTKVDIK

SEQ ID NO: 27 - DNA, 357 bp, Artificial Sequence
  Humanized 5D3-5 - heavy chain
caagttcaactggtgcagagcggtccggaagttaaaaaaccgggtgcaagcgttcgtctgagctgtaaagcaagcggttatgcatttaataccagctggatgaattgggttcgtcaggcacctggtcaaggtctggaatggattggtcgtatttatcctggtgatggtgataccaactataacggtaaattcaaaggtaaagcaaccatgaccgcagataaaagcaccaataccgcatatatggaactgcgtagcctgcgtagtgatgataccgcagtgtattattgtggtcgtggtgaatggtatctgtactattttgattattggggtcagggcaccctggtcaccgtctcctca

SEQ ID NO: 28 - DNA, 321 bp, Artificial Sequence
  Humanized 5D3-5 - light chain
gacatccagatgacacagagcccgagcagcgttagcgcaagcgttggtgatcgtgttaccattagctgtagcgccagccagggtattaacaattttctgacctggtatcagcagaaactgggtaaagcactgcaactgctgatctattataccagcagcctgcatagcggtgttccgagccgttttagcggtagcggtagtggcaccgattataccctgaccattagcagtctgcaaccggaagattttgcaacctattattgtcagcagtatagcaatctgccgtttacctttggtccgggtacaaaagtcgatatcaaa

SEQ ID NO: 29 - protein, 750 aa, Homo sapiens
  Isoform PSMA-1
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 30 - protein, 119 aa, Artificial Sequence
  Consensus VH
  VARIANT 30: Gly, Ala, Val, Leu, Ile, Tyr, His, Lys, Arg, Gln, Asn, Glu, Asp, Ser or Thr
  VARIANT 31: Pro or Thr
  VARIANT 32: Gly, Ala, Val, Leu, Ile, Tyr, His, Lys, Arg, Gln, Asn, Glu, Asp, Thr or Ser
  VARIANT 34: Met, Ile or Leu
  VARIANT 55: Gly, Ala, Val, Leu, Ile, Tyr, His, Gln, Asn, Glu, Asp, Ser or Thr
  VARIANT 56: Gly, Ala, Val, Leu, Ile, His, Lys, Arg, Gln, Asn, Glu, Asp, Ser or Thr
  VARIANT 57: Gly, Ala, Val, Leu, Ile, Tyr, His, Gln, Asn, Glu, Asp, Ser or Thr
  VARIANT 58: Gly, Ala, Val, Leu, His, Gln, Asn, Glu, Asp, Ser or Thr
  VARIANT 61: Gly, Ala, Val, Leu, Ile, Tyr, His, Lys, Arg, Gln, Asn, Glu, Asp, Ser or Thr
  VARIANT 62: Gly, Ala, Val, Leu, Ile, Tyr, His, Lys, Arg, Gln, Asn, Glu, Asp, Ser or Thr
EVQLVESGGGLVQPGGSLRLSCAASGYAFXXXWXNWVRQAPGKGLEWISRIYPGXXXXNYXXKFKGKATISADKSKNTLYLQMNSLRAEDTAVYYCARGEWYLYYFDYWGQGTLVTVSS

SEQ ID NO: 31 - protein, 107 aa, Artificial Sequence
  Consensus VL
  VARIANT 30: Gly, Ala, Val, Leu, Ile, Tyr, His, Lys, Arg, Gln, Asn, Glu, Asp, Ser or Thr
  VARIANT 31: Gly, Ala, Val, Leu, Ile, Tyr, His, Lys, Arg, Gln, Asn, Glu, Asp, Ser or Thr
DIQMTQSPSSLSASVGDRVTITCSASQGIXXFLTWYQQKPGKALKLLIYYTSSLHSGVPSRFSGSGSGTDYTLTISSLQPEDFATYYCQQYSNLPFTFGQGTKVEIK

SEQ ID NO: 32 - protein, 10 aa, Artificial Sequence
  Consensus VH CDR 1
  VARIANT 5: Gly, Ala, Val, Leu, Ile, Tyr, His, Lys, Arg, Gln, Asn, Glu, Asp, Ser or Thr
  VARIANT 6: Pro or Thr
  VARIANT 7: Gly, Ala, Val, Leu, Ile, Tyr, His, Lys, Arg, Gln, Asn, Glu, Asp, Thr or Ser
  VARIANT 9: Met, Ile or Leu
GYAFXXXWXN

SEQ ID NO: 33 - protein, 16 aa, Artificial Sequence
  Consensus VH CDR2
  VARIANT 6: Gly, Ala, Val, Leu, Ile, Tyr, His, Gln, Asn, Glu, Asp, Ser or Thr
  VARIANT 7: Gly, Ala, Val, Leu, Ile, His, Lys, Arg, Gln, Asn, Glu, Asp, Ser or Thr
  VARIANT 8: Gly, Ala, Val, Leu, Ile, Tyr, His, Gln, Asn, Glu, Asp, Ser or Thr
  VARIANT 9: Gly, Ala, Val, Leu, His, Gln, Asn, Glu, Asp, Ser or Thr
  VARIANT 12: Gly, Ala, Val, Leu, Ile, Tyr, His, Lys, Arg, Gln, Asn, Glu, Asp, Ser or Thr
  VARIANT 13: Gly, Ala, Val, Leu, Ile, Tyr, His, Lys, Arg, Gln, Asn, Glu, Asp, Ser or Thr
RIYPGXXXXNYXXKFK

SEQ ID NO: 34 - protein, 11 aa, Artificial Sequence
  Consensus VL CDR1
  VARIANT 7: Gly, Ala, Val, Leu, Ile, Tyr, His, Lys, Arg, Gln, Asn, Glu, Asp, Ser or Thr
  VARIANT 8: Gly, Ala, Val, Leu, Ile, Tyr, His, Lys, Arg, Gln, Asn, Glu, Asp, Ser or Thr
SASQGIXXFLT